(19) United States
(12) Patent Application Publication
(10) Pub. No.: US 2024/0130882 A1
(43) Pub. Date: Apr. 25, 2024

(54) INTELLIGENT ORTHOPEDIC DEVICE SYSTEM, AND METHOD OF USING THE SAME

(71) Applicant: Medflexion Health, Inc., San Marcos, CA (US)

(72) Inventors: John R. Bond, San Marcos, CA (US); Alan Heim, Vista, CA (US)

(21) Appl. No.: 18/382,502

(22) Filed: Oct. 20, 2023

Related U.S. Application Data
(60) Provisional application No. 63/418,390, filed on Oct. 21, 2022.

(51) Int. Cl. A61F 5/01; A61F 7/00; A61N 1/04; G16H 20/30

(52) U.S. Cl. CPC A61F 5/0118; A61F 7/00; A61N 1/0456; G16H 20/30; A61F 2007/0035

(57) ABSTRACT

Aspects of the present disclosure generally pertain to an orthopedic device that monitors a user's orthopedic position. Aspects of the present disclosure more specifically are directed toward an intelligent orthopedic device, system, and method where an assessment of the user's current state can be determined, monitored, measured, and addressed. The device may be adapted for various parts of the user's body. The system and method may interface the device with a computational device via a communication network to access and exploit sensor data provided by the device.

### CROSS-REFERENCE TO RELATED APPLICATION

This application claims priority to U.S. provisional patent application 63/418,390 filed Oct. 21, 2022 and entitled INTELLIGENT BRACE SYSTEM AND METHOD OF USING THE SAME, the contents of all of which are incorporated herein by reference in their entirety.

### BACKGROUND OF THE INVENTION

### Technical Field

The present disclosure generally pertains to orthopedic devices, and is more particularly directed towards orthopedic devices or orthotics that monitor a user's orthopedic position.

### Related Art

The human body may develop musculoskeletal issues where jointed members are statically or dynamically misaligned. This may be due to body dynamics or misuse. Orthopedic devices are designed to address musculoskeletal issues. Some areas of interest here may include upper and lower extremities, the trunk, and the head, having anatomical jointed areas including head-cervical spine, thoracic spine-thorax, lumbar spine, shoulder/brachium, elbow-forearm, wrist-hand, pelvis-hip-femur, knee-tibia, ankle-foot, etc. Further, each area may have its own, unique musculoskeletal issues.

In some cases, one may merely take corrective action once becoming aware, such as “sitting up straight” after being told or remembering not to improperly operate a computer mouse, for example. In other cases, orthopedic devices such as braces may be worn. In general, braces properly align, correct position, support, stabilize, and protect certain parts of the body and in particular muscles, joints, and bones. Braces can be used as a preventive measure to reduce the likelihood of a future injury. Braces can also be used to correct harmful behaviors to avoid painful surgery. Alternately, braces can be used post injury or surgery to assist in the healing process and for rehabilitation purposes. Generally, a consumer can obtain a brace from their overseeing medical professional or over the counter.

For the most part, current braces are outdated. They are typically made out of a flexible fabric, for a specific body part and unlike a cast are removable. Generally, braces do not provide any metrics such as the time in the brace is use or the events that occurred while in use. They can be either too rigid preventing any movement at all such as a foot “boot” or conversely, too flexible in that the user would be unaware of their excessive movement until there is pain or further damage. Additionally, a patient may need a variety of braces over the course of time or treatment.

U.S. Pat. No. 9,808,208 issued to Erman on Nov. 7, 2017, shows a device, system and method for monitoring carpal tunnel syndrome (“CTS”). The device comprises a body configured to be worn by a user, sensors, a processor, a vibration mechanism, and a power source. The sensors monitor a position of the user's hand to prevent CTS. The processor of the device is configured to determine if the user's hand is in a CTS position and the processor is configured to generate an alert signal to alert the user to the CTS position.

U.S. Pat. No. 10,188,346 issued to Erman on Jan. 29, 2019, shows a device, system and method for monitoring cubital tunnel syndrome (“CuTS”). The device comprises a body configured to be worn by a user, sensors, a processor, a vibration mechanism, and a power source. The sensors monitor a position of the user's upper arm and forearm to prevent CuTS. The processor of the device is configured to determine if the user's upper arm and forearm is in a CuTS position and the processor is configured to generate an alert signal to alert the user to the CuTS position.

U.S. Pat. No. 10,874,347 issued to Erman on Dec. 29, 2020, shows a series of multi-plane sensors are attached to different points on the body such as forearms, calves, lower backs, and necks and are used to establish planes of each body part is disclosed herein. The multi-plane sensors stream the plane data and velocity vector data to a centralized computing device such as a computer, smart phone, or smart watch. The centralized computing device runs a software application which includes an open source library which platform partners utilize to write proprietary applications which utilize the sensor stream data.

The present disclosure is directed toward overcoming known problems and problems discovered by the inventor. Further, the present disclosure addresses deficiencies in performance in known orthopedic devices.

### SUMMARY OF THE INVENTION

Aspects of the present invention generally pertain orthopedic devices that monitor a user's orthopedic position. Aspects of the present invention more specifically are directed toward an intelligent orthopedic device and system where an assessment of the user's current state can be determined, monitored, measured, and addressed.

An intelligent orthopedic device for a jointed body part of a user, where the jointed body part includes first member and a second member joined together by a joint, is disclosed herein. The intelligent orthopedic device includes a body mount configured to attach to the user proximate the jointed body part of the user, a body sensor coupled to the body mount, where the body sensor is configured to provide an angular displacement between the first member and the second member of the jointed body part, a position resistor coupled to the body mount, and a controller coupled to the body mount and communicably coupled to the body sensor. The position resistor is configured to apply a force to the angular displacement to the jointed body part. The controller is configured to receive the angular displacement from the body sensor and to operate the position resistor to apply the force opposite the angular displacement to the jointed body part.

According to one embodiment, an intelligent orthopedic device system for a jointed body part of a user, where the jointed body part includes a first member and a second member joined together by a joint, is also disclosed herein. The intelligent orthopedic device system includes the abovementioned intelligent orthopedic device and an offboard computing device communicably coupled to the controller of the intelligent orthopedic device, and configured to host and run a related software application. The software application is programmed to receive and send information regarding one or more features and functions of the intelligent orthopedic device including at least one of measuring, monitoring, tracking and storing the angular displacement between the first member and the second member of the jointed body part.

According to another embodiment, an intelligent orthopedic device for a jointed body part of a user, the jointed body part including first member and a second member joined together by a joint, is also disclosed herein. The intelligent orthopedic device includes a body mount configured to attach to the user proximate the jointed body part of the user, a body sensor coupled to the body mount, where the body sensor is configured to provide an angular displacement between the first member and the second member of the jointed body part, a means to apply at least one of cooling, warming, and Transcutaneous Electrical Neuromuscular Stimulation (“TENS”) to the jointed body part of the user, were said means is coupled to the body mount, and a controller coupled to the body mount and communicably coupled to the body sensor. The controller is configured to receive the angular displacement from the body sensor, to operate said means, and to apply said at least one of cooling, warming, and “TENS” to the jointed body part of the user via said means.

### BRIEF DESCRIPTION OF THE DRAWINGS

FIG. 1 is a schematic diagram of a generalized intelligent orthopedic device, according to one exemplary embodiment of the present disclosure.

FIG. 2 is an illustration of an exemplary controller for an intelligent orthopedic device configured to be worn by a user or attached to a body part of interest of the user, according to one embodiment of the present disclosure.

FIG. 3 is a schematic diagram of internal components of the controller and sensor electronics for an intelligent orthopedic device, according to one exemplary embodiment of the present disclosure.

FIG. 4 is a top view of controller and sensor electronics for an intelligent orthopedic device, according to one exemplary embodiment of the present disclosure.

FIG. 5 is a bottom view of the controller and sensor electronics of FIG. 4.

FIG. 6 illustrates various anatomical misalignments of a human hand, for reference.

FIG. 7 is a first perspective view of an exemplary intelligent orthopedic device, according to one embodiment of the present disclosure.

FIG. 8 is a second perspective view of view of the exemplary orthopedic device, of FIG. 7.

FIG. 9 is a third perspective view of view of the exemplary orthopedic device, of FIG. 7.

FIG. 10 illustrates an exemplary orthopedic device in use, according to one embodiment of the present disclosure.

FIG. 11 is a schematic diagram of an integrated controller and sensor for an intelligent orthopedic device, according to one exemplary embodiment of the present disclosure.

FIG. 12 is an illustration of an exemplary intelligent orthopedic device configured to be worn on a hand of a user, according to one embodiment of the present disclosure.

FIG. 13 is a schematic diagram of a top view of an intelligent orthopedic device, according to one exemplary embodiment of the present disclosure.

FIG. 14 is a schematic diagram of a side view of the intelligent orthopedic device of FIG. 11.

FIG. 15 illustrates various anatomical motions of a human shoulder, for reference.

FIG. 16 illustrates various anatomical motions of a human arm, for reference.

FIG. 17 illustrates various anatomical motions of a human leg, for reference.

FIG. 18 illustrates various anatomical motions of a human foot, for reference.

FIG. 19 block diagram illustrating functional and operational features of an intelligent orthopedic device, according to one embodiment of the present disclosure.

FIG. 20 is an illustration of an intelligent orthopedic device system, according to one embodiment of the present disclosure.

FIG. 21 an intelligent orthopedic device system in use, according to one embodiment of the present disclosure.

FIG. 22 is a smart phone screen shot of a user display for an intelligent orthopedic device system, according to one embodiment of the present disclosure.

FIG. 23 is a smart phone screen shot of a user display for an intelligent orthopedic device system, according to one embodiment of the present disclosure.

FIG. 24 is a smart phone screen shot of a user display for an intelligent orthopedic device system, according to one embodiment of the present disclosure.

FIG. 25 is a smart phone screen shot of a user display for an intelligent orthopedic device system, according to one embodiment of the present disclosure.

FIG. 26 is a smart phone screen shot of a user display for an intelligent orthopedic device system, according to one embodiment of the present disclosure.

FIG. 27 is a smart phone screen shot of a user display for an intelligent orthopedic device system, according to one embodiment of the present disclosure.

FIG. 28 is a computer screen shot of a user display for an intelligent orthopedic device system, according to one embodiment of the present disclosure.

FIG. 29 is a computer screen shot of a user display for an intelligent orthopedic device system, according to one embodiment of the present disclosure.

### DETAILED DESCRIPTION

The detailed description set forth below in connection with the appended drawings is intended as a description of presently-preferred embodiments of the disclosure and is not intended to represent the only forms in which the present disclosure may be constructed or utilized. The description sets forth the functions and the sequence of steps for constructing and operating the disclosure in connection with the illustrated embodiments. It is to be understood, however, that the same or equivalent functions and sequences may be accomplished by different embodiments that are also intended to be encompassed within the spirit and scope of the disclosure.

As used herein, the following terms and variations thereof have the meanings given below, unless a different meaning is clearly intended by the context in which such term is used. The terms “a,” “an,” and “the” and similar referents used herein are to be construed to cover both the singular and the plural unless their usage in context indicates otherwise. As used in this disclosure, the term “comprise” and variations of the term, such as “comprising” and “comprises,” do not exclude other components or steps.

For purposes of this application, the term “medical professional” is defined to include any one or more persons who can be a physical therapist, physician, physician's assistant, a medical practice or clinic, and/or any person(s) qualified to perform an exam and provide a diagnosis to a user or patient. This may be, in some embodiments, a single licensed medical professional; however, in another embodiment, it may include multiple licensed medical professionals, technicians, assistants, nurse practitioners, and/or other persons qualified under state regulations, guidelines, and/or standard industry practice.

The terms “computer,” “computer device,” and “server” as used herein, refers to a device and/or system of devices that include at least one computer processing element, e.g., a central processing unit (CPU), and some form of computer memory having a capability to store data. The computer may comprise hardware, software, and firmware for receiving, storing, and/or processing data as described below. For example, a computer or computer device may comprise any of a wide range of digital electronic devices, including, but not limited to, a server, a desktop computer, a laptop, a smart phone, a tablet, or any form of electronic device capable of functioning as described herein. The term “database” as used herein, refers to any form of one or more (or combination of) relational databases, object-oriented databases, hierarchical databases, network databases, non-relational (e.g. NoSQL) databases, document store databases, in-memory databases, programs, tables, files, lists, or any form of programming structure or structures that function to store data as described herein. The term “computer memory” as used herein refers to any tangible, non-transitory storage that participates in providing instructions to a processor for execution. Such a medium may take many forms, including but not limited to, non-volatile media, volatile media, and any equivalent media known in the art. Non-volatile media includes, for example, ROM, magnetic media, and optical storage media. Volatile media includes, for example, DRAM, which typically serves as main memory. Common forms of computer memory include, for example, hard drives and other forms of magnetic media, optical media such as CD-ROM disks, as well as various forms of RAM, ROM, PROM, EPROM, FLASH-EPROM, solid state media such as memory cards, and any other form of memory chip or cartridge, or any other medium from which a computer can read. While several examples are provided above, these examples are not meant to be limiting, but illustrative of several common examples, and any similar or equivalent devices or systems may be used that are known to those skilled in the art.

Aspects of the present disclosure generally pertain to orthopedic devices or orthotics, capable of monitoring a user's orthopedic position. As used herein, “orthopedic” and “orthopedic devices” may refer to medical and/or non-medical devices, systems, treatments, etc. Aspects of the present disclosure more specifically are directed toward “smart orthotics” or an intelligent orthopedic device, system, and method where an assessment of the user's current state can be determined, monitored, measured, and addressed. Further, some of the disclosed embodiments may include addressing the assessment of the user's state in real time (e.g., with correction, user/3^{rd} party notification, comfort, therapy/treatment, etc.).

Aspects of the present disclosure are also directed to an intelligent orthopedic device such as an intelligent brace system and method of using the same. In particular the intelligent orthopedic device or intelligent brace system and method of using the same may dynamically provide bracing in response to the user exceeding a predefined threshold. For example, in one preferred embodiment, the system may include a brace worn by a user that normally remains flexible or otherwise relaxed, and only stiffens when the user misaligns his/her body.

Further, the intelligent orthopedic device may use a wired and/or wireless means of communicating with an offboard computing device that hosts a related software application. The software application may be programmed to receive and send information regarding the features and functions of the orthopedic device including, but not limited to, measuring, monitoring, tracking and storing the movements, therapeutic features and biometric data of the user. The information received from the brace may be stored in a cloud platform which can then be reviewed and analyzed to assist in the recovery of the user. The information stored in the cloud may also be provided via the web to a remote portal where a third-party can also review the device information, and/or it can be further processed to extract additional information and meaning. An interface on the computing device may provide for the user to engage with the orthopedic device, adjust the parameters, and/or review any stored information. The user and/or their healthcare provider may assess the information to make informed decisions.

According to one exemplary embodiment, the intelligent orthopedic device may address issues affecting the wearer, such as musculoskeletal issues. For example, as a brace the orthopedic device may be adjusted (e.g., in stiffness) as the user's progress either improves or worsens. In even further embodiments the brace may include therapeutic properties that can improve the wearer's comfort, experience, and/or even healing process, such as by instructing appropriate physical therapy exercises, providing heating and/or cooling, and/or providing transcutaneous electrical nerve stimulation (TENS).

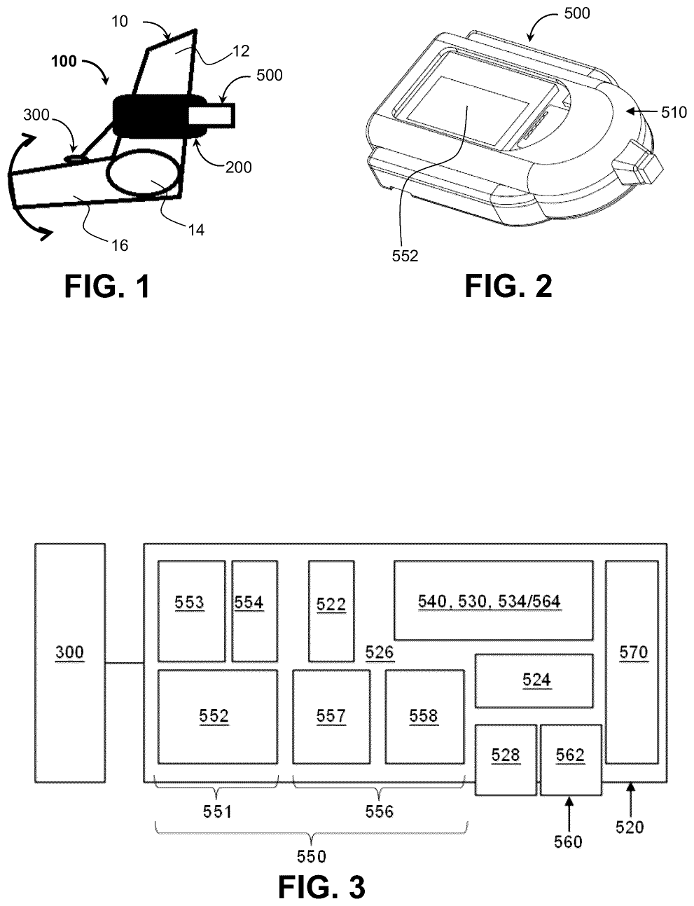

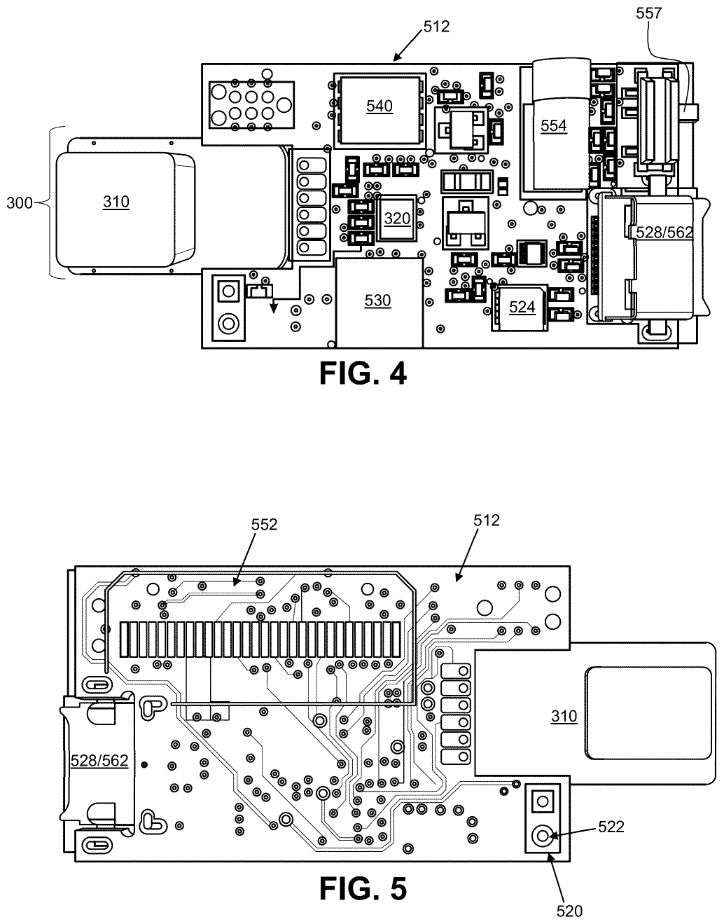

FIG. 1 is a schematic diagram of a generalized intelligent orthopedic device, according to one exemplary embodiment of the present disclosure. As disclosed herein, an intelligent orthopedic device or smart orthotics (hereinafter “orthopedic device” 100) may address musculoskeletal issues related to the upper and lower extremities, the trunk, and the head, and their anatomical jointed areas (see e.g., FIG. 4 and FIGS. 13-16). For example, and as illustrated, the generic body part of interest of a user/wearer 10 may generally have a base member 12 (e.g., an upper arm, a forearm, a thigh, etc.), a jointed region or joint 14 (e.g., elbow, wrist, knee, spine, etc.), and a dynamic member 16 (e.g., a forearm, a hand, a calf, etc.).

It is understood that the name or type of member (i.e., base, joint, dynamic) may vary based on perspective, for example, the forearm may be viewed as a base member when addressing musculoskeletal issues related to the wrist, but viewed as a dynamic member when addressing musculoskeletal issues related to the elbow. Further, this generalized language and illustration is understood as non-limiting, but rather illustrative of the general concept disclosed herein. For example, in one embodiment, the disclosed orthopedic device 100 may address musculoskeletal issues across the spine (or portions thereof), when may generally be viewed as multi-jointed region.

In keeping with this generalized language and illustration, the disclosed orthopedic device 100 may generally include a body mount 200, at least one body sensor 300, and a controller 500 communicatively coupled to the at least one body sensor 300. Preferably at least one of the body sensor 300 and the controller 500 are coupled to (or removably couplable to) the body mount 200, which in turn can be attached to area of interest the user/wearer 10. The body mount 200 may be adapted to mount on or otherwise attach to the particular area of interest using any conventional means, such as garments, harnesses, sleeves, straps, adhesives, etc., or combination thereof. Further, The body mount 200 may be directly mounted (e.g., worn by the user) or indirectly mounted (e.g., attached to clothing) to the user 10. In one preferred embodiment, the present disclosure comprises a brace or a body to be worn by a user. Preferably, the body mount 200 will be form-fitting, or otherwise closely position the one or more body sensors 300 proximate the user's body, so as to aid in providing more precise measurements.

The body sensor(s) 300 may be configured to determine a position of the dynamic member 16, relative to the base member 12, for example, and communicate this information to the controller 500. In particular, body sensor(s) 300 may be configured to an angular displacement metric between the base member 12 and the dynamic member 16 of the body part of interest, preferably precise micro measurements (e.g., including but not limited to 1 degree variations). It is understood that the metrics may be in any convenient form, and may be converted on or off the body sensor(s) 300 (e.g., using a separate processor). One or more of the body sensor(s) 300 may configured to determine additional relevant information, such as environmental conditions. As above, one or more of the body sensors 300 may be coupled to the body mount 200, mounted directly or indirectly to the user 10 (e.g., via adhesive, strap, clothing, etc.), or any combination thereof. Further, the body sensor(s) 300 may be communicably coupled to the controller 500 directly (e.g., wired, fiber, conduit, etc.), indirectly (e.g., wireless communications, inductively, capacitively, visually, magnetically, etc.), or any combination thereof.

FIG. 2 is an illustration of an exemplary controller for an intelligent orthopedic device configured to be worn by a user or attached to a body part of interest of the user, according to one embodiment of the present disclosure. As above, the controller 500 may be communicatively coupled (e.g., wired, wirelessly, etc.) to the body sensor(s) 300 (FIG. 1) and removably couplable to the body mount 200 (FIG. 1), which in turn may be directly or indirectly couplable to the user/wearer 10 (FIG. 1).

According to one embodiment, the elements of the controller 500 may be configured as a single electronic unit. In particular, the controller 500 may include a housing 510, such as a conventional case made of a rigid material that houses and protects the electronics of the controller 500 as a modular unit. The housing 510 of the controller 500 may be adapted for a particular use or environment, as needed. For example, the structure of housing 510 may be adapted to be water proof, water resistant, impact resistant, etc. Also for example, the housing 510 may be made of non-reactive or hypoallergenic materials.

According to one preferable embodiment, the housing 510 of the controller 500 may include a toolless couple to the body mount 200. For example, the toolless couple may include any combination of an overall form-factor that is configured to mate with a pocket or other receiver embedded in the body mount 200 (FIGS. 7-9), a mechanical quick release couple configured mate with a reciprocal couple of the body mount 200 (FIG. 10), or any other conventional toolless couple.

In operation, the controller 500 may be configured to instigate action or issue commands in response to received sensor information (e.g., body position), as discussed below. For example, controller 500 may be configured measure, process, record, and/or transmit the information to the user (e.g., for information and/or responsive action), to a medical professional (e.g., for monitoring and/or treatment), or to a backend server (e.g., for remote processing and or/for individual and/or group data analytics). Also for example, the controller 500 may be configured determine whether any musculoskeletal issues are occurring and/or oncoming, and may address them such as via alerting, instructing, and/or interacting with the user 10, discussed further below. Also for example, the controller 500 may be configured provide the sensor information to an offboard processor or computer (e.g., backend server, local computer program, phone app, etc.), which is capable or more capable to determine whether any musculoskeletal issues are oncoming and/or occurring and to provide the user with appropriate feedback.

FIG. 3 is a schematic diagram of internal components of the controller and sensor electronics for an intelligent orthopedic device, according to one exemplary embodiment of the present disclosure. As above, the orthopedic device 100 (FIG. 1) may include the body sensor(s) 300 and the controller 500 physically coupled to (or removably couplable to) the body mount 200 (FIG. 1). Likewise, the body sensor 300 may include one sensor (or a plurality of sensors making up a sensor array) communicably coupled to the controller 500. Generally, the controller 500 may include conventional computational and electronic equipment such as a power supply 520, a processor 530, and a memory 540.

Generally, the power supply 520 may be configured as an offboard power supply (e.g., plug in power), an onboard energy storage 522 (e.g., battery, rechargeable battery, etc.), or combination thereof (e.g., battery electronically coupled to a charging circuit 524). Where the power supply 520 utilizes offboard power, the power supply 520 may further include an offboard power port 528 (e.g., DC power plug, USB power/data port, etc.). Depending on the onboard energy storage 522 used, the offboard power port 528 may also accessible so that the onboard energy storage 522 can connected to a remote computing device to either transfer information or to recharge the onboard energy storage 522. Alternately, the onboard energy storage 522 may be configured as an inductively charged battery such that the housing 510 of the controller 500 may be generally sealed or even waterproofed.

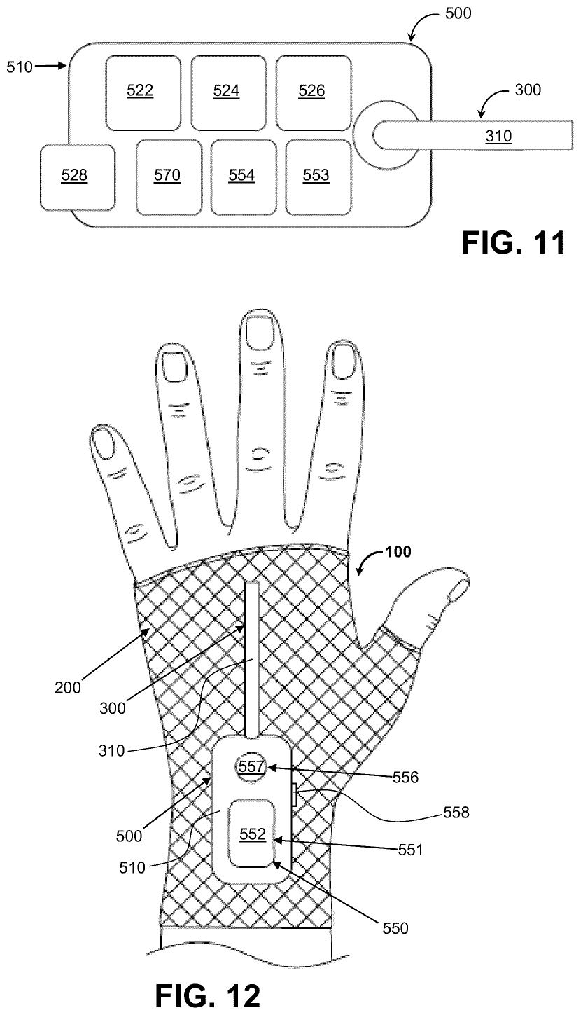

The power supply 520 may be configured to power any onboard electronics (e.g., processor 530 and memory 540), using any conventional power control circuitry 526 (FIG. 11). In addition, the power supply 520 may be further configured to power offboard electronics where applicable (e.g., an offboard body sensor 300), again using any conventional power control circuitry 526.

Generally, the processor 530 may be configured to provide any instructions, processing power, and commands the controller 500 required to carry out its tasks. In particular, the processor 530 may be configured to operate, and to measure, monitor and track real-time movements of the user 10 (FIG. 1) via its communications with the sensor 300. Further, the processor 530 may be configured to record, compare, and analyze movement or other metrics over time in combination with the memory 540. Depending on the configuration, the computing board (e.g., PCB assembly) may also house traces, wires and a least one circuit board interface configured to communicate with the one or more sensors. As a computational device, it is understood that the processor 530 may be a single processor (e.g., SoC), a combination of processors (e.g., CPU, GPU, VPU, NPU, ASIC, FPGA, etc.), or a combination thereof. In a preferred embodiment the processor 530 may include a microprocessor with embedded wireless technology.

Generally, the memory 540 may be programmed to with operation instruction sets and applications to run on the processor 530. The memory 540 may be further programmed to set limits or thresholds for the user's position and/or movement. In addition, position and movement can be calculated (via the processor 530) and stored by using data from a single body sensor 300, or combined data from a multitude of sensors. Further, the memory 540 or portions thereof may be writeable such that parameters may be adjusted as needed and/or desired. According to one embodiment, memory 540 may be integrated into the processor 530 as an internal memory where measurement data can be stored and/or be communicably coupled to an external memory for the storage logging of data, for example when a connection to an external computing system is not available.

According to one embodiment, the controller 500 may include additional electronic equipment besides the power supply 520, processor 530, and memory 540. In particular, the controller 500 may further include at least one of a user interface 550, a communication port 560, and a controller output 570. Generally, the controller output 570 may be configured to communicate commands, power, and/or energize external features of the orthopedic device 100 (e.g., heat, cooling, stiffening, stimulation, etc.), discussed further below.

Generally, the user interface 550 (or “U/I”) may include a user output 551 configured to signal the user visually, aurally, haptically, etc., and/or may include a user input 556. Generally, the user input 556 (“U/I input”) may be configured receive information from the user 10 using any conventional interface. For example the U/I input 556 may incorporate any conventional user interface including, but not limited to, manual interfaces such as buttons (e.g., device operation control 557 configured as an on/off switch), touch screens, biometric readers, as well as touchless interfaces such as microphones, nearfield readers, and cameras, to name a few.

Similarly, the user output 551 (“U/I output”) may be configured communicate alert signals to the user 10 when the body part of interest is not in the correct position or has otherwise exceeded a preset threshold limit (or limits). For example, the U/I output 551 may include a display 552 (FIG. 2) such as an OLED display, and the alert signal can include a visual alert such as a light, text, image, and/or other indicator shown on the display 552. As above, the display 552 may be a touch screen that also serves as the U/I input 556. Also for example, the U/I output 551 may include an audio output 553 such as a speaker or other audio device, and the alert signal can include an audio alert such as a ‘ping’, bell, worded statement, or other audible indicator. Also for example, the user output 551 may include a haptic device 554 such as vibration motor or other technology that outputs a tactile response to the user 10 that is associated with the occurrence of a predefined event or other indication. Preferably, the haptic device 554 will be located locally on the device (e.g., haptic motor enclosed in the housing 510), so that even if the user 10 does not see or hear the audio or visual alerts (e.g., emanating from an offboard computing device such as a cell phone), the user 10 would still feel a vibration emanating from the controller 500. As discussed below, aspects of the U/I output 551 may incorporated into an offboard computing device such as a smart phone running an associated application.

Generally, the orthopedic device 100 may be configured to communicate information about the position of the user's body part to a remote computing device, for example, running an application software, and where the information collected can be stored in a cloud-based platform and can be reviewed and analyzed. The software program can also be used to adjust the features of the device as further detailed below. As such, the communication port 560 may be configured to communicate with an offboard computing device. In particular, the communication port 560 may include a wired connection (e.g., USB plug), a wireless connection (e.g., RF radio, NFC radio, optical/capacitive/inductive couple, etc.), or any combination thereof. For example, the communication port 560 may include a USB-C connection configured communicate data and to act as the offboard power port, and further include a wireless radio configured to communicate sensor data, commands, and other information to one or more offboard computing devices (e.g., a user's mobile device, a backend server, a medical professional's computer, etc.)

According to one preferred embodiment, the processor 530 may include a microprocessor with embedded wireless technology or otherwise in communication. Examples of wireless technology may include WiFi, BT, BTLE, UWB, or NFC, which are well known in the art. Similarly, the processor 530 may also include the necessary memory and interfaces to communicate with a sensor or sensors, store measurement data, and monitor system status such as battery charge level, which are similarly well-known in the art. Accordingly, the processor may be configured to communicate with a computing system such as a laptop, desktop, tablet, mobile device or smartwatch that is running a software program that has algorithms that measure, monitor and provide feedback regarding the information collected from the processor. The information can then be used and analyzed in more detail.

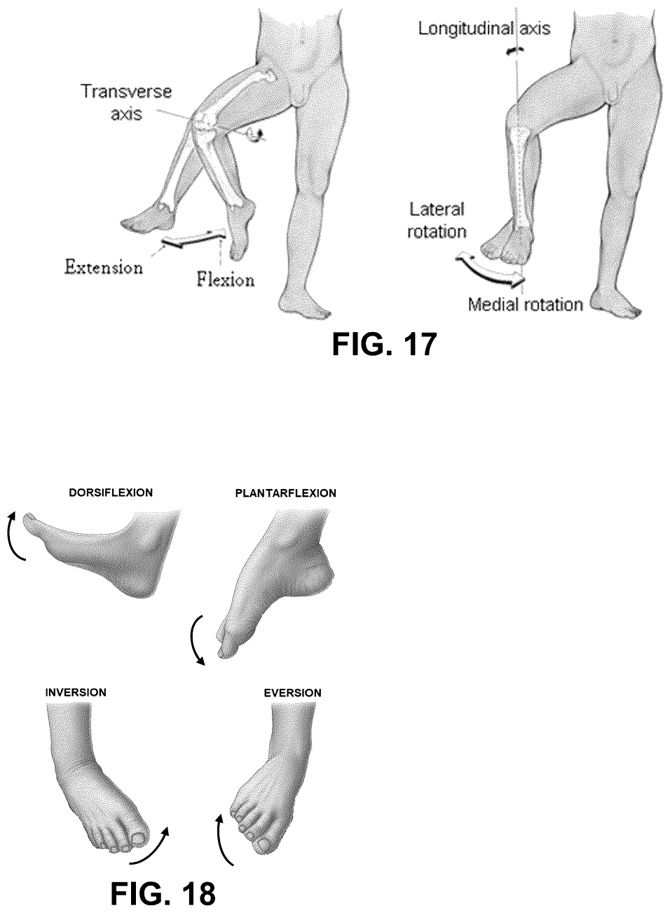

FIG. 4 is a top view of controller and sensor electronics for an intelligent orthopedic device, according to one exemplary embodiment of the present disclosure. FIG. 5 is a bottom view of the controller and sensor electronics of FIG. 17. As above, the elements of the controller 500 may be configured as a single electronic unit within the housing 510. In particular, and as shown, elements of the controller 500 may populate a circuit board 512, which is then installed into the housing 510.

Above, the circuit board 512 may provide any traces, wires, vias, interfaces, etc., as well as power control circuitry 526 (FIG. 11) needed to provide power and communications within the controller, as well as to other elements of the orthopedic device 100 (FIG. 1). For example, in the illustrated architecture, the top side of the circuit board 512 serves as a support platform for and connector between various electronic components, such as the charging circuit 524 (USB charger) and the offboard power port 528 (USB-C plug) of the power supply 520, the processor 530 (microcontroller/CPU), the memory 540 (solid state), the U/I output 551 (haptic motor 554) and U/I input 556 (device operation control 557 configured as on/off switch) of the user interface 550, to name a few. Also for example, the bottom side of the circuit board 512 serves as a support platform for and connector between various electronic components, such as the energy storage 522 (showing battery connector-battery removed for clarity) of the power supply 520 and the wired external communication port 562, in this particular architecture.

Further, the circuit board 512 may be configured to provide any structure, connections, and/or electronics needed to provide power and communications with other elements of the orthopedic device 100. In particular, in the illustrated architecture, the circuit board 512 may serve as a support platform for and connector between the controller 500 and at least one body sensor 300. For example, the circuit board of the controller 500 may structurally and electronically coupled to a bend sensor 310 and an inertial measurement unit (IMU) 320, as well as others. Thus, the body sensor 300 is both electronically coupled to the circuit board the controller 500 (i.e., powered by power supply 520), and communicably coupled with the processor 530. It is understood that, depending on the nature of the sensor(s) 300, it may be completely or only partially enclosed within the housing 510.

According to one preferred embodiment, the bend sensor 310 may include a capacitive bend sensor. For reference, there are three common types of bend sensors, namely piezoelectric, piezoresistive and capacitive types. While piezoresistive types may provide adequate measurements for general applications, the more costly capacitive bend sensors have higher stability, sensitivity and resolution. Also, while the piezoelectric type are less costly, they may be susceptible to humidity. Importantly, the inventor has discovered that accurately measuring micro-movements by the orthopedic device 100 can provide terabytes of precise continuous data, which might otherwise go unregistered or filtered out as noise. For example, some of the continuous data that can be gathered might include Range of Motion, Motion Direction, Activity Duration, Therapy Utilization, Compliance Data, Use of Recovery Protocols, Events, Correlations, Benchmarking, Step Tracking. Moreover, this data may then be made available for advanced processing, and can later be used for future AI opportunities. The inventor has further discovered that harmful micro-motions can be modified using the orthopedic device 100, as discussed below. Accordingly, the bend sensor 310 may configured to measure micro-movements using one or more capacitive bend sensors.

According to one embodiment, the sensor 300 may include an array or variety of other sensors. To illustrate, a gyroscope is an angular velocity sensor which is commonly used for measurement of human posture and movement. Flexible angular sensor is operated by measuring change of electrical output or displacement with respect to angular change. Electromagnetic tracking system is a three-dimensional measurement device that has been used in human posture and movement which consists of a transmitter and receivers. A low-frequency magnetic field is generated by the transmitter and detected by the receivers. The positions and orientations of the receiver relative to the transmitter can then be calculated by the system.

According to one embodiment, one or more of the body sensor(s) 300 may be configured to sense and measure a particular aspect of the user's position. In particular, the body sensor 300 (or sensors) may be configured to monitor the position of the user's body part and its movement. For example, at least one (or more than one) sensor 300 may be used to measure and monitor the movement or position of the body part. To illustrate, using the bend sensor 310 combined with the IMU 320, the controller 500 (via its processor 320) may determine the both a bend angle of the joint of interest, as well as it's dynamic character. Optional sensors that can be used are accelerometers, inertial measurement units (IMUs), force sensors, strain sensors, or angular sensors, gyroscope, flexible angular sensor, electromagnetic tracking system, optical sensors, and sensing fabrics. In a preferred embodiment the body sensor 300 may include electronic positional sensors or angular sensors, which may be used by the controller 500, as they can be used in various clinical applications including the analyses of general physical activity, gait, posture, trunk and upper limb movement.

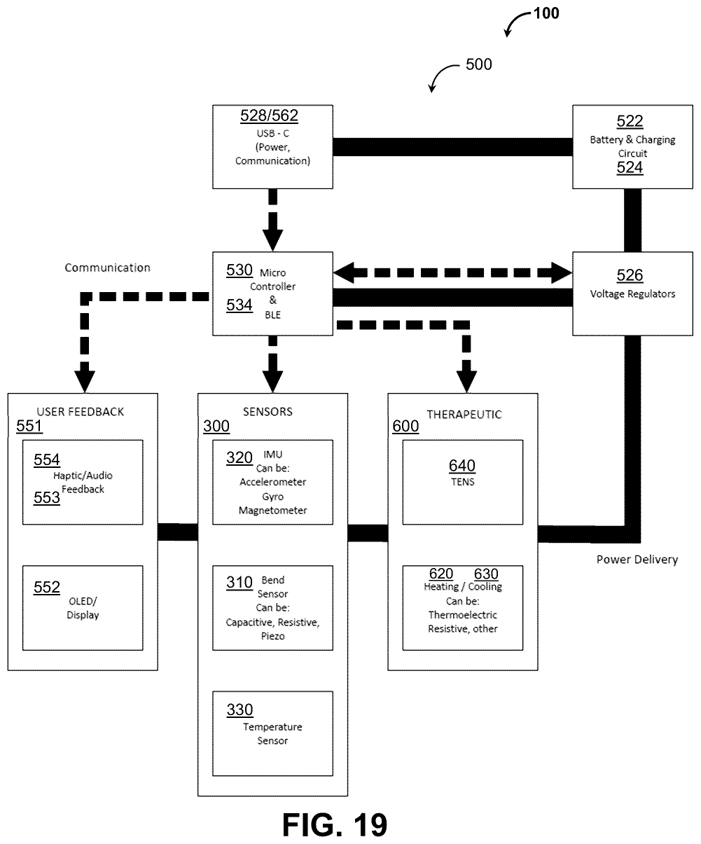

As above, the sensor 300 may include an array or variety of body sensors 300 configured to determine any relevant information of the user and/or the user's environment (e.g., environmental conditions). Also as above, one or more of the body sensors 300 may be coupled to the body mount 200, mounted directly and/or indirectly to the user 10, and may be communicably coupled to the controller 500 directly and/or indirectly to the processor 530. For example, one or more of the body sensors 300 may be configured to monitor and track other information about the user's identity or vitals such as pulse, oxygenation, heart rate, hydration, temperature, etc. More specifically, additional sensors 330 (FIG. 19) may include body sensors 300 configured as biometric sensors, pulse Ox sensors, heart rate sensors, hydration sensors, temperature, sensors etc. It is understood that, depending on the nature of the additional body sensor 330, an additional sensor user interface may be included as well.

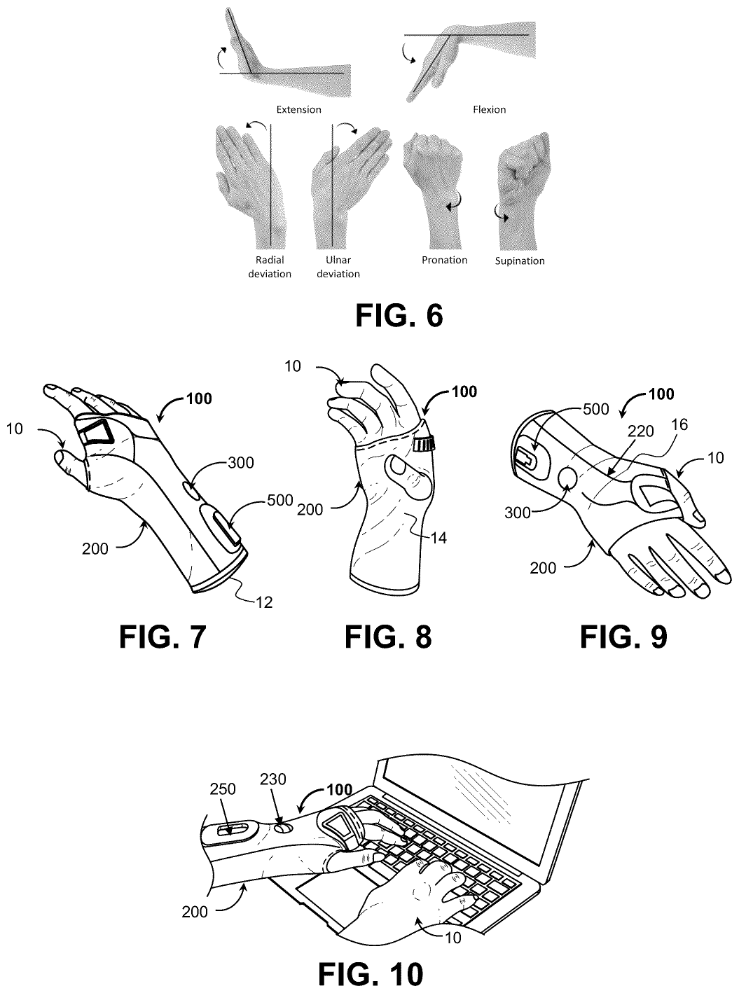

FIG. 6 illustrates various anatomical misalignments of a human hand, for reference. As shown the human hand may be misaligned in a variety of ways. While this may not have a negative impact on a person over short periods, extended periods could lead to discomfort or worse. Carpal tunnel syndrome (“CTS”) is the collection of symptoms and signs associated with median neuropathy at the carpal tunnel. Most CTS is related to idiopathic compression of the median nerve as it travels through the wrist at the carpal tunnel. Frequently, people spending extended periods working on a computer will rely on braces to properly align, correct position, support, stabilize, and protect their wrists over extended periods. Unfortunately, braces also inhibit intermittent misalignment, which may be useful for a task, and braces themselves may create discomfort over extended periods. As above, the device of U.S. Pat. No. 9,808,208 issued to Erman, monitors a position of the user's hand and sends alert signal to the user.

FIG. 7 is a first perspective view of an exemplary intelligent orthopedic device, according to one embodiment of the present disclosure. FIG. 8 is a second perspective view of view of the exemplary orthopedic device, of FIG. 7. FIG. 9 is a third perspective view of view of the exemplary orthopedic device, of FIG. 7. Here, the body part of interest of the user/wearer 10 is the wrist, and the orthopedic device 100 is specifically adapted as dynamic, intelligent wrist brace. As above, the orthopedic device 100 may include the body mount 200, one or more of the body sensors 300, and the controller 500.

The body mount 200 of the device is preferably a flexible material. Some of the most common materials used for braces are neoprene, polyester, and nylon. Some braces may also have hinges and may feature some metal or hard plastic components for fastening or to provide additional support. The fabric or material used can depend on the body part being monitored. For example, the body mount 200 can be configured be worn on a body part such as the wrist as shown, or alternately on an elbow, shoulder, knee, ankle or back. The size and cut of the body mount 200 can be modified for comfort and is generally removable from the body part either by sliding it on or off like a sleeve or by using Velcro, snaps or some other strap so the user can wear the device when desired or required and remove it at other times.

According to one embodiment, and as illustrated, the body mount 200 of the orthopedic device 100 may be configured as a wrist brace or otherwise worn across the user's wrist. In particular, the body mount 200 may be made of a textile fabric or other flexible material adapted to mount or otherwise anchor itself to the wearer's forearm (base member 12) and hand (dynamic member 16 dynamic member 16) and span across the wearer's wrist (jointed region or joint 14). Further, the body mount 200 may include any conventional features of a wrist brace, such as being fingerless or open finger, washable fabric, size adjustment, elastic fit, etc.

According to one embodiment, the body mount 200 may include a sensor interface 230 configured to receive the one or more of the body sensors 300. In particular, the sensor interface 230 may be configured to support and position the body sensor 300 in a predetermined position relative to the body part of interest (here across the user's wrist). For example, the sensor interface 230 may include a pocket or channel in the fabric of the body mount 200. This may be particularly useful with the abovementioned bend sensor, as it may spread across a greater region of the body for greater precision in angular measurement. In addition, by locating all, or even part, of the body sensor 300 within the body mount 200 (and outside of the controller 500), the controller 500 may be made more compact.

According to another embodiment, the sensor interface 230 may be configured to allow the user to remove the body sensor 300, such as a pocket opening or flap cover, which may be useful for cleaning of the body mount 200 or repair/adjustment/replacement of the body sensor 300. Beneficially, the user 10 may also be able to use the body mount 200 as a brace without the body sensor 300 installed.

Similarly, according to one embodiment, the body mount 200 may include a controller interface 250 configured to receive the controller 500. In particular, the controller interface 250 may be configured to support and position the controller 500 in a predetermined position relative to the body part of interest (here across the user's wrist). For example, the controller interface 250 may include a pocket or channel in the fabric of the body mount 200. Further, the controller interface 250 may be configured to allow the user to remove the controller 500, such as a pocket opening or flap cover, which may be useful for cleaning of the body mount 200 or repair/adjustment/replacement or recharging of the controller 500. Beneficially, the user 10 may also be able to use the body mount 200 as a brace without the controller 500 installed.

According to one embodiment, the orthopedic device 100 may be configured to use knitted stretch fabric sensors. In particular, the body sensor 300 may be directly integrated into the fabric of the body mount 200. The operational concept of this type of sensor is to measure the changes of resistance in knitted strips. It gives a linearly increasing asymptotic resistance when it is stretched, up to almost maximum stretch developed a minimally intrusive wearable system consisting of a Lycra leotard with conductive polymer strain sensors chemically deposited in selected areas. Other combinations of materials and polymers were used to optimize the response time of the sensors. In this embodiment, the body sensor 300 may be arranged such that the knitted stretch fabric sensor detect the posture/position and/or movement of the wearer.

Further, it is believed that polymeric conductors and semiconductors may offer several advantages with respect to metal and inorganic conductors: lightness, large elasticity and resilience, resistance to corrosion, high flexibility, impact strength, etc. Because of its elasticity, ergonomic comfort, and high piezoresistive and thermoresistive coefficients, the combination of polypyrrole as conducting polymer and Lycra/cotton as fabric is particularly effective.

FIG. 10 illustrates an exemplary orthopedic device in use, according to one embodiment of the present disclosure. Here, the user is only using one orthopedic device 100 and leaving the other hand free. Further, the user has removed the controller 500 (FIG. 9) of the orthopedic device 100.

As above, the controller 500 may be configured to removably attach to the body mount 200 of the orthopedic device 100. In particular, the controller interface 250 of the body mount 200 may be configured as a toolless couple to the housing 510 (FIG. 2) of the controller 500. For example, the controller interface 250 may include a mechanical quick release couple configured mate with a reciprocal couple of the housing 510 of the controller 500, or any other conventional toolless couple.

According to one embodiment, the body mount 200 may be configured to remain stiff in one or more areas. In particular, the body mount 200 may include one or more stiffeners 220 (e.g., a rigid insert like a splint or rod) insertable in positions or areas of interest, for example to prevent certain movements. As such, the body mount 200 may include one or more pockets or sleeves configured to receive the stiffeners 220, such that the rigid inserts can be slid into the pocket/sleeve of the body mount 200, remain in place, and be sufficiently supported to transfer loading between the user 10 and the stiffeners 220. This feature may be desirable, for example, for use during sleep, so that the user is unable to have freedom of movement when unconscious. In an alternate embodiment, the stiffener(s) 220 may be attached to an external surface of the body mount 200.

FIG. 11 is a schematic diagram of an integrated controller and sensor for an intelligent orthopedic device, according to one exemplary embodiment of the present disclosure. As above, the controller 500 may be communicably coupled to the body sensor(s) 300 directly or indirectly, or any combination thereof. Here, the controller 500 may be configured as a modular unit (including its housing 510) together with the body sensor(s) 300.

According to the illustrated embodiment, the body sensor 300 and controller 500 may be integrated together and configured as a single, integrated electronic unit. In particular, the housing 510 of the controller 500 may be configured to house, support, and protect the electronics of both the controller 500 and the body sensor 300. For example, and as illustrated, the housing 510 may enclose, the energy storage 522, the charging circuit 524, the power control circuitry 526, the offboard power port 528, the audio output 553, the haptic output device 554 (e.g., vibration), and the controller output 570, to name a few. Further, the housing 510 of the controller 500 may be a case or other body that also houses at least one body sensor 300 together with the components of the controller 500. In this way, the body sensor 300 may be structurally and electronically coupled to the circuit board the controller 500 (i.e., powered by power supply 520), within the housing 510 of the controller 500, and communicably coupled directly with the processor 530, also within the housing 510.

Similarly, and as shown, at least one body sensor 300 may both be mounted to the housing 510 of the controller 500, and extend therefrom, such that its communication couple and power is protected within the housing 510, while its sensing element(s) (e.g., flex sensor) may interface directly, or more directly, with the user 10 (e.g., being routed through a pocket or channel in the body mount 200). Beneficially, the integrated controller and sensor may be conveniently powered, installed, and removed, as a single unit, such as for cleaning, repair, upgrading, etc.

FIG. 12 is an illustration of an exemplary intelligent orthopedic device configured to be worn on a hand of a user, according to one embodiment of the present disclosure. As above, the orthopedic device 100 may be configured to be worn across the user's wrist, and may include the body mount 200, one or more of the body sensors 300, and the controller 500. Also as above, the body sensor(s) 300 and the controller 500 may be integrated together as a single module. Also as above, the controller 500 may include a user interface 550, including at least one of the U/I output 551 and at least one U/I input 556.

According to one embodiment, the U/I output 551 of the controller 500 may be configured to communicate at least one of: alerts, status information (e.g., of the wearer, of the orthopedic device 100, or other relevant status), and instructions (discussed below) to the user 10. For example, the controller 500 may be configured to communicate alerts to the user 10 when the body part of interest (here, the wrist) has exceeded a preset threshold limit(s) or is otherwise is not in a correct position. According to one embodiment, the U/I output 551 configured to communicate alert signals may be located on the an exposed surface of the controller 500. For example, an alert signal can be a visual alert such as an indicator light (e.g., LED) or a light on the display 552.

According to one embodiment, the U/I output 551 may be embedded within the controller 500. For example, an alert signal may be an audio alert, such as a ping sounded by the audio output 553 (e.g., from within the in the housing 510). Also for example, the alert signal to the user 10 may be a vibration that is energized by the haptic output 554 (also embedded in the housing 510). Beneficially, even if the user 10 does not see or hear the audio or visual alerts, the user 10 would feel a vibration in the orthopedic device 100.

According to one embodiment a method of using the orthopedic device 100 may include, in response to a sensed or otherwise measured/monitored metric, issue an alert to the user 10. For example, if the body part of interest has exceeded or is not in the desired or selected position, the user may be notified using, for example, the U/I output 551. Alternately, if the user 10 is using an offboard computing device such as a cell phone running a mobile application in connection with the orthopedic device 100, the visual, audio or vibrating signal would occur on the phone via the app.

As above, the alert can come in many forms and can be a visual alert, an audio alert or vibration. For example, the entire controller 500 may light up if an alert is triggered or a small light can appear on the display 552 of the controller 500. If the alert is a vibration alert, a vibration motor (haptic output 554) may also be housed in the controller 500. An audio alert may also be used, which like the vibration motor, the audio output 553 may be housed in the controller 500. Initially, the software associated with processor 530 uses preset parameters or thresholds to alert the user 10 of prohibited movements. Generally the threshold or movement limits may be set based on the severity of the user's pain. For example, a person experiencing more pain may have more restrictions that a person experiencing less pain. These parameters can be adjusted or custom set for each user 10 either manually or by a physician, for example. When the threshold or limit is triggered because a of a prohibited action, the user 10 is alerted either visually, audially or through vibration. Over time, with multiple alerts the user 10 may re-learn new behavior.

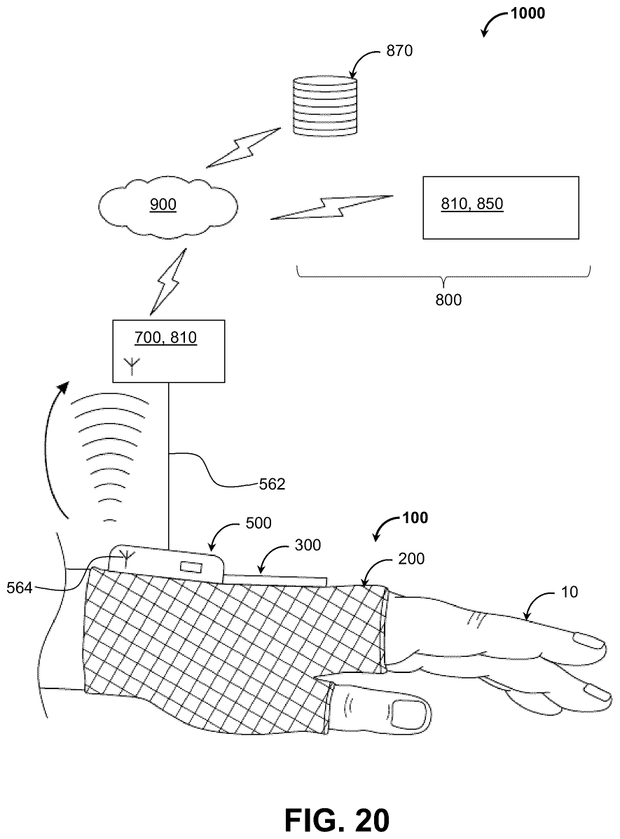

According to one embodiment, and as discussed further below, the user 10 may be alerted by a remote user interface, which is apart from of the orthopedic device 10. For example, if the user 10 were wearing the orthopedic device 10 while typing on a keyboard for work as in FIG. 12, a screen may pop up on the computer telling the user 10 they have reached or exceeded the threshold limit. Beneficially, the alerts can also be used to garner compliance. For example, alerts can be designed to remind the user 10 to perform their assigned exercises. Alerts can also be managed (e.g., via an algorithm) to instruct a patient to “take a break” from the present activity because it may be harmful. The system 1000 (FIG. 20) can also use both the orthopedic device 100 and remote user interface alerts together. By using one or more of these alert features, the user 10 may benefit over time by stopping potentially harmful movements.

According to one embodiment, the controller 500 may be configured for multi-mode communications with the user. In particular, the U/I output 551 of the controller 500 may be configured to communicate a plurality of: alerts, status information (e.g., of the wearer, the orthopedic device 100, etc.), and/or instructions (discussed below), with one or more being communicated via a distinct mode. For example, and as above, the U/I output 551 may include the display 552 that is viewable from its exterior, as well as the audio output 553 and the haptic output 554 embedded in the housing 510. Each of the display 552, the audio output 553, and the haptic output 554 can provide the user 10 with same information about the orthopedic device 100 (e.g., power, connection status, battery life and alerts as described below) via different modes. In this way, the orthopedic device 100 may simultaneously communicate to the user 10 in alternate ways (i.e., parallel or many-to-one communications). Beneficially, where one mode is not effective (e.g., audio in a noisy environment) the user may still be notified via vibration.

According to one embodiment, the different modes may be configured to indicate a different type of message (i.e., serial or one-to-many communications). For example, a vibration signal may be associated with or otherwise indicate an adjustment reminder/“nudge”, whereas a bell/beeping signal may be used to indicate a warning or more serious condition.

According to one embodiment, the controller 500 may be more interactive with or tailorable by the user 10. In particular, the U/I input 556 of the controller 500 may include any number of device operation controls 557 and/or include one or more of the user feedback controls 558. Further, this may be in addition to the abovementioned U/I outputs 551.

The device operation controls 557 may range from a basic “on/off” button for the entire the orthopedic device 100 to a selector for each of its individual features. As discussed below, the orthopedic device 100 may be configured to communicate with an offboard computing device (e.g., application running on cell phone), which may already have a richer, more extensive user interface available. Accordingly, it may be preferable that the controller 500 have more limited device operation controls 557 onboard, such as power, volume, intensity, and the like, with further functionality provided with the offboard computing device.

Similarly, the user feedback controls 558 may range from a single “event indicator” button to a more interactive communication interface (e.g., allowing the user to respond to prompts presented by the controller 500). As above, since the orthopedic device 100 may be configured to communicate with an offboard computing device (i.e., having a richer, more extensive user interface), it may be preferable that the controller 500 have more limited user feedback controls 558 onboard, such as being limited to a pain and/or event button, a variable comfort level indicator, and the like.

It is possible that the default thresholds will not be appropriate for each user. For example, the user 10 may experience pain before reaching the threshold limit. Thus, in an optional embodiment, the user feedback controls 558 of the controller 500 may include a button configured to indicate to the controller 500 that the user just experienced pain. Preferably the button to indicate pain (“pain button”) may be located in a readily available location on the exterior of the controller 500 (e.g., located on or near the display 552). Further, the processor 530 may be programmed to record any use of the “pain button” along with any other available data of interest.

According to one embodiment, user feedback controls 558 may be configured to initiate a predetermined response. In particular, if the user 10 presses an “event” button (similar or the same as the “pain button”), the controller 500 may be configured to respond by recording that the event occurred (or that pain was experienced), to respond with remedial action, and/or to respond with other appropriate action. For example, the controller 500 may output a threshold-related indication on the display 552, and if a user experienced pain while performing an action, the software could record this incident, as well as any other available data of interest and shut down any activity of the orthopedic device 100 that might be associated the pain (or event). Advantageously, the user 10 or healthcare provider might be able to review this to determine whether any preset limits were not correct, and/or may need to be adjusted.

As illustrated, the orthopedic device 100 may include the bend sensor 310 of the body sensor(s) 300 extending from the controller 500. It should be understood, the bend sensor 310 may be located internally, externally, and/or embedded in the body mount 200. Notwithstanding, or alternately, according to one embodiment, the sensor may be physically separated from the controller 500. For example, the body mount 200 may be comprised of a sensing fabric, as discussed above, so as to function as bend sensor 310. Also for example, the bend sensor 310 may be a separate unit, attached to the user via any conventional means.

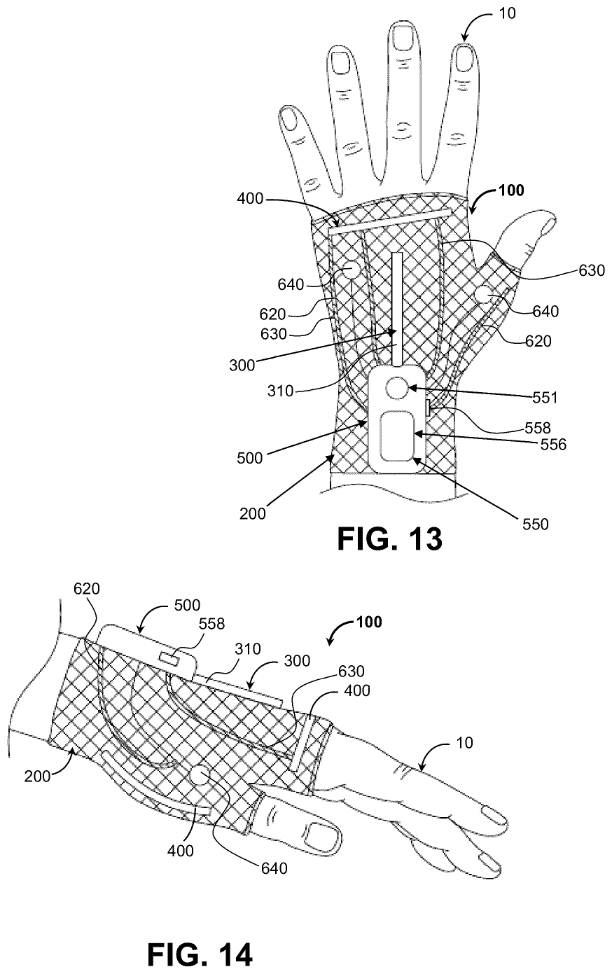

FIG. 13 is a schematic diagram of a top view of an intelligent orthopedic device, according to one exemplary embodiment of the present disclosure. FIG. 14 is a schematic diagram of a side view of the intelligent orthopedic device of FIG. 11. As above, the orthopedic device 100 may be configured to be worn across the user's wrist, and may include the body mount 200, one or more of the body sensors 300, and the controller 500. Also as above, the body sensor(s) 300 and the controller 500 may be integrated together as a single module with the user interface 550, including at least one of the U/I output 551 and the U/I input 556. Further, the U/I output 551 and the U/I input 556 may be configured to communicate with the user 10 via multiple modes.

As discussed above, the disclosed orthopedic device 100 may be configured to measure, monitor, and ultimately determine or otherwise assess a current state of the user. In addition, the disclosed orthopedic device 100 may be configured to act upon the user 10, or otherwise address the user's state. In particular, the orthopedic device 100 may be further configured to power and operate features that resist said state, sooth an affected area of interest, and/or guide the user in a specific response. Further, the response the user's state may be in real time, at a scheduled time, or preventively (e.g., based on any measured indications).

According to one embodiment, the orthopedic device 100 may include a position resistor 400. In particular, the position resistor 400 may be configured to provide a counter force against the body part of interest once a predefined threshold is met. Further, elements of the position resistor 400 can be located on the body mount 200 where the user needs the most stability or support. For example, the body sensor 300 may include the bend sensor 310 configured to measure a bend angle in the user's wrist (e.g., Extension, Flexion, Radial/Ulnar deviation, Pronation, and/or Supination) and continuously report said bend angle to the processor 530.

Upon meeting a predetermined threshold stored in the memory 540 (e.g., Flexion beyond n-degrees), the controller 500 may operate the position resistor 400 to the adjust the stiffness of the orthopedic device 100, or alternately to apply a force opposite the predetermined threshold. For example, the position resistor 400 may be in communication with the processor 530 so that if the user reached the preset limit, the orthopedic device 100 could immediately stiffen preventing further movement.

According to one embodiment, the position resistor 400 may be an electromechanical stiffener. In particular, the electromechanical stiffener may be set across a bend axis, and left in a “normally relaxed” or flexible state. Then the predefined threshold in met, the controller 500 may energized it so that it stiffens.

For example, one or more the position resistors 400 may be attached to the body mount 200 at the back of the hand, and oriented substantially perpendicular to the wrist's normal axis of rotation with regard to Extension/Flexion. Also for example, and as shown, one or more the position resistors 400 may be attached to the body mount 200 along other locations of interest, such as across the knuckles or along the user's thumb.

Under normal conditions, the position resistors 400 may be left in their de-energized, flexible states, however, upon being energized, they will stiffen and resist rotation in both directions, thus straightening out the user's wrist or other areas of interest. Alternately, the position resistors 400 may be only partially energized (e.g., pulsed) so as to communicate to the user that the predetermined threshold has been met.

According to one embodiment, the electromechanical stiffener may be a rod or tube structure, where rod or tube changes shape and/or rigidity when an electrostatic interaction occurs. If electromechanical stiffeners are used, the rod or tube would be in communication with or otherwise operable by the processor 530 (e.g., via the controller output 570). Alternately, the position resistor 400 may be configured to operate via alternate energizing mechanisms (e.g., pneumatic, hydraulic, magnetic, etc.).

According to one preferred embodiment, the orthopedic device 100 may operate through use of electrorheological (ER) fluids housed in one or more channels or cavities in the body mount 200. ER fluids are composed of electrically polarizable particles in an insulating medium. Electrorheological fluids (ERF) change viscosity when an electric field or current is applied. A special type of ERF consists of polyurethane particles, which, when a current is applied, the fluid stiffens. Thus, the controller 500 may apply a current to the position resistors 400 via the controller output 570, causing the orthopedic device 100 to stiffen and prevent unwanted movement. In application, the processor 530 may be programmed to prefer a predefined position, and if the body part or its movement is outside of the position the ER fluid may be activated, changing the stiffness of the brace

According to one embodiment, the orthopedic device 100 may be configured to be “active” or “reactive”. In particular, the position resistor(s) 400 may vary in stiffness based the on preset movement limits. For example, the electromechanical stiffener or ER Fluids increase in resistive force the further or faster the user exceeds any predetermined threshold(s). Beneficially, the stiffening achieved by the electromechanical rods, tubes or ER fluid can be used as not only a form of an “alert” or correction, but also potentially extend the life of the orthopedic device 100. In this way, the orthopedic device 100 may be used to inform the user of a misalignment, or even correct the misalignment.

According to one embodiment, the stiffness of the orthopedic device 100 may be adjustable. In particular, the orthopedic device 100 may vary the number of channels energized by the controller output 570. For example, by providing numerous channels, the position resistor 400 has the capability to act more like a cast, whereas by providing numerous channels minimal channels the position resistor 400 would not ever become too rigid. Similarly, the controller 500 may be configured to energize a full set, a minimal set, or even a select set of channels via the controller output 570 so as to vary the “stiffness” of the orthopedic device 100 and/or to target a particular area for stiffening/flexibility.

According to one embodiment, the stiffness of the orthopedic device 100 may be adjusted or otherwise varied either automatically or manually. In particular, the preset limits may change over time as the use of the ER fluids or electromechanical stiffener can make the orthopedic device 100 very firm of stiff in the beginning of treatment and then more flexible by the end of treatment. As such, the controller 500 may be adapted to dynamically adjust stiffness (e.g. via adaptive learning, sensor feedback, manual input, etc.).

As above, the orthopedic device 100 may be configured treat or sooth an affected area of interest. In particular, the orthopedic device 100 may include a therapeutic add-ons such as means 600 (FIG. 19) to apply at least one of temperature control and/or neuromuscular stimulation. Further, the treatment period can be set for a certain period of time, for example, either by the user or a medical professional, or manually as needed for pain or comfort.

According to one embodiment, the orthopedic device 100 may include a body warmer 620. In particular, the body warmer 620 may include one or more conventional warming elements routed through body mount 200 and arranged so as to be in thermal communication with the body part of interest. Further, the body warmer 620 may be coupled to the controller output 570 and configured so as to be energized or otherwise powered by the controller 500, and controlled by the processor 530. In this way, the processor 530 can be programmed to produce an increase in temperature in the body warmer 620 such that the body part is also warmed.

According to one embodiment, the body warmer 620 may include a radiating wire embedded in the body mount 200 of the orthopedic device 100, where the electric coils produce heat upon receiving current from the controller output 570. Alternately, the body warmer 620 may include a mesh of Polyester filament and Micro Metal Conductive Fiber folded into a protective Polyimide Film. Alternately, the body warmer 620 may include a carbon fiber heating material. Preferably, the body warmer 620 may include electric coils embedded in the body mount 200 of the orthopedic device 100, where the electric coils produce heat upon receiving current from the controller output 570.

According to one embodiment, heat may be provided preemptively. In particular, heat may be applied to the user's body before an exercise is performed. Beneficially, the heat increases blood flow and metabolic activity which leads to a loosening of muscle tissue. Similarly, warming an injured area relaxes stiffness and relieves pain in aching joints, such as those affected by arthritis. The duration and temperature of the heat may be adjusted according to any combination of predefined algorithms stored software on the memory 540, real time feedback provided to the processor 530, and learned modifications also stored on the memory 540. Additionally, the system 1000 may store all of the data related to use of the warming feature and can relay this information to the user and medical professional.

According to one embodiment, the orthopedic device 100 may include a body cooler 630. In particular, the body cooler 630 may include one or more conventional cooling elements routed through body mount 200 and arranged so as to be in thermal communication with the body part of interest. Further, the body cooler 630 may be coupled to the controller output 570 and configured so as to be energized or otherwise powered by the controller 500, and controlled by the processor 530. In this way, the processor 530 can be programmed to produce a decrease in temperature in the body cooler 630 such that the body part is also cooled. Preferably, the body cooler 630 may be electrically powered through the use of a Peltier device embedded in the body mount 200 of the orthopedic device 100. A Peltier device is preferable, as a Peltier junction uses electrical current to produce a temperature differential which can cause both heating and cooling of the surface that is thermally to the user. As such, the body cooler 630 may provide cooling upon receiving current from the controller output 570. In operation, the controller 500 may be configured to provide cooling, for example, post exercise to reduce inflammation and swelling, and/or to provide comfort and soothing.

### [TENS]

According to one embodiment, the orthopedic device 100 may include a body stimulator 640. In particular, the body stimulator 640 may include one or more conventional Transcutaneous Electrical Neuromuscular Stimulation (“TENS”) elements or electrodes anchored to and positioned about the body mount 200. Further, the electrodes of the body stimulator 640 may be arranged so as to be in electrical communication with the body part of interest. Thus, along with other forms of treatment from the orthopedic device 100, the user 10 can receive electrical stimulation as part of the treatment. Beneficially, TENS is a physical therapy treatment used to manage short- and long-term pain in physical therapy, and can be therapeutic and aid in recovery. Further, applying an electrical current can help relax and/or strengthen muscles, block pain signals, and improve blood circulation.

In operation, the processor 530 (through software) may be programmed to produce an electric therapeutic shock to a desired location on the body. In particular, the processor 530 may power the body stimulator 640 via the controller output 570 and deliver an electrical impulse directed to a location on the body. For example, a TENS circuit may be fixed to the body mount 200 and energized via wired connection to the controller 500. Like the thermal treatment features, the processor 530 can be programmed to adjust the intensity of the electrical impulse sent via the controller output 570. Similarly, the processor 530 can be programmed to adjust the profile and duration of the electrical impulse treatment.

According to one embodiment, the orthopedic device 100 may store all of the data related to use of TENS locally and/or remotely. Further, and as with other captured data, the orthopedic device 100 may relay this information to the user 10 and/or a medical professional, for example, via the communication port 560 of the controller 500.

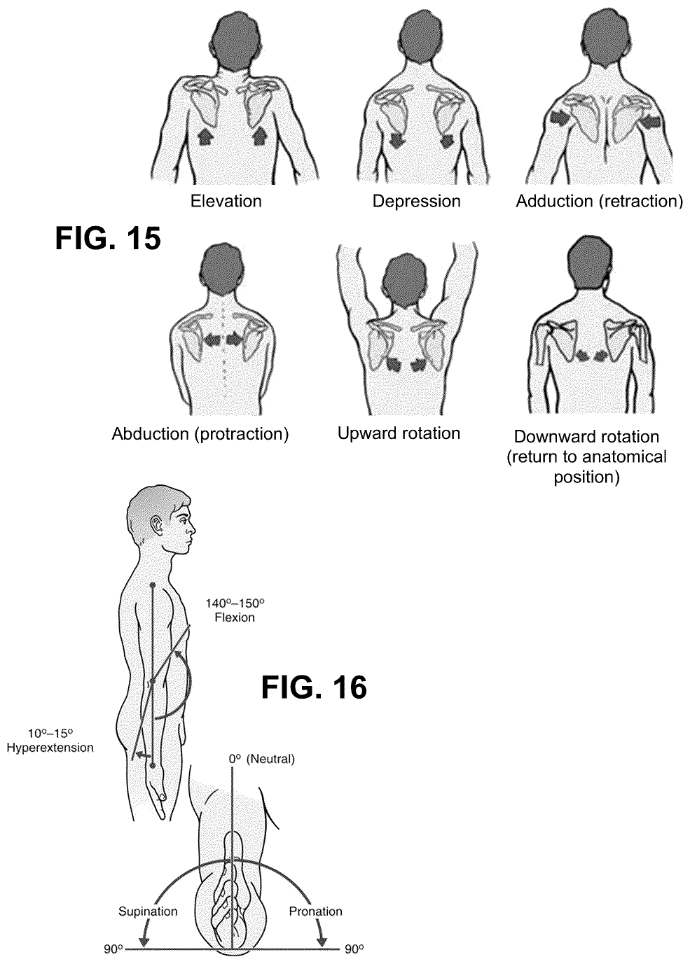

FIG. 15 illustrates various anatomical motions of a human shoulder, for reference. FIG. 16 illustrates various anatomical motions of a human arm, for reference. FIG. 17 illustrates various anatomical motions of a human leg, for reference. FIG. 18 illustrates various anatomical motions of a human foot, for reference. As discussed above, it is understood that the orthopedic device 100 may adapted to many areas of interest and across many articulations and movements. As such, there may be many uses for the orthopedic device 100, beyond addressing movements related to CTS and CuTS. Further, the orthopedic device 100 may be adapted to dynamically address misalignments for each area using continuous monitoring of even micromotions, and applying user alerts, user instruction, and resistance in response, as well as treatments such as thermal and electrical impulse treatments.

While many uses of the orthopedic device 100 may be for comfort and soothing, the orthopedic device 100 may also be applied to both chronic and acute conditions. For example, it is understood that the orthopedic device 100 (and system 1000) may be applicable to Osteoarthritis, Rheumatoid arthritis, Juvenile arthritis, Back problems—bad posture, scoliosis, degenerative disc disease, Gout, Osteoporosis, Osteopenia, Carpal tunnel syndrome, Fibromyalgia, Epicondylitis (Tennis Elbow), Cubital Tunnel Syndrome, to name a few. Also for example, it is understood that the orthopedic device 100 (and system 1000) may be applicable to Fractures, Sprain or torn tendons or ligaments, Broken bones, Dislocations, Tendonitis, Bursitis, also to name a few.

FIG. 19 block diagram illustrating functional and operational features of an intelligent orthopedic device, according to one embodiment of the present disclosure. As illustrated and as discussed above, the orthopedic device 100 may include the body mount 200, one or more of the body sensors 300, and the controller 500. Via power and communication control, the controller 500 may operate the overall function of the orthopedic device 100, including but not limited to power delivery (using e.g., voltage regulators, the battery and the charging circuit), offboard power and communications (using the offboard communication and power ports—e.g., USB type), onboard processing and wireless communications (e.g., using the micro controller with embedded wireless radio), onboard sensor communications (e.g., imu accelerometer, gyro, magnometer; bend sensor-capacitive, resistive, piezo; and temperature sensor), local user communications (e.g., user feedback-haptic/audio feedback; and OLED display), and therapeutic response (e.g., tens; and heating/cooling-thermoelectric resistive, other).

FIG. 20 is an illustration of an intelligent orthopedic device system, according to one embodiment of the present disclosure. As above, the orthopedic device 100 may include the body mount 200, one or more of the body sensors 300, and the controller 500, which may operate the overall local function of the orthopedic device 100. Also as above, the orthopedic device 100 may be configured to measure, monitor, assess a current state of the user, and then address the user's state (e.g., resist said state, sooth or treat an affected area of interest, and/or guide the user in a specific response). As shown here, the orthopedic device 100 may be incorporated into, and be central to, an intelligent orthopedic device system (hereinafter “system”) 1000.

Generally, the system 1000 may include the orthopedic device 100 and an offboard computing device 700 to which it is communicatively connected. According to one embodiment, the offboard computing device 700 may be directly coupled to the orthopedic device 100 via wired connection to the wired external communication port 562 of the controller 500 (not shown). This may be useful for charging the energy storage 522 (e.g., where wired external communication port 562 is a USB type connection), downloading data from the memory 540, and/or in compromised wireless environments.

Preferably, and as illustrated, the orthopedic device 100 may be wirelessly communicably coupled to the offboard computing device 700 via the wireless external communication port 564 of the controller 500 over a wireless communication network. In particular, wireless external communication port 564 of the orthopedic device 100 may be configured to communicate with the offboard computing device 700 over a relatively short range network, such as a Wireless Body Area Network (WBAN), a Wireless Personal Area Network (WPAN), or a Wireless Local Area Network (WLAN). Accordingly, the wireless external communication port 564 may include a radio configured to communicate over, for example, Bluetooth, WiFi, etc. with the offboard computing device 700.

According to one embodiment, the offboard computing device 700 may include a computing device such as a laptop or desktop computer, a tablet, mobile phone or smartwatch that is both configured to communicate with the orthopedic device 100 and to host and run a related software application. The software application may be programmed to receive and send information regarding the features and functions of the orthopedic device 100 including, but not limited to, measuring, monitoring, tracking and storing the movements, therapeutic features and biometric data of the user 10. Further, the offboard computing device 700 may include a conventional, full featured user interface that is capable of providing for the user to engage with the orthopedic device 100, adjust the parameters, and/or review any stored information, for example via the software application.

According to one embodiment, the system 1000 may further include at least one remote computing device 800 and a cloud network 900. The remote computing device 800 may include a computing device such as a laptop or desktop computer, a tablet, mobile phone configured to communicate with the offboard computing device 700 over the cloud network 900. Further, the remote computing device 800 may be embodied as a personal device 810 (e.g., a device operated by the user 10), a third party device 850 (e.g., a device operated by the user's medical professional), and/or a backend server 870 (e.g., a device configured for cloud storage, data analytics, machine learning, artificial intelligence (“AI”), etc.).

The backend server 870 may be configured to execute machine-readable instructions referred to herein as a software application. The backend server 870 may include a database, and the backend server 870 may be further configured to generate, in the database, a user account. The backend server 870 exists in the cloud, and thus may be accessible via standard internet protocols. The backend server 870 may be further configured to manage account data and medical data retained in the database, namely sensor data and owner information as discussed in greater detail below. According to one embodiment, the software application is accessible via the internet using a web browser on a computer device.

In the system 1000, the offboard computing device 700 may generally operate as a wireless interface between the orthopedic device 100 and the cloud network 900. For example, the offboard computing device 700 may merely act as a communications link for the orthopedic device 100, which may then communicate directly across the cloud network 900. Also for example, the orthopedic device 100 may merely act as a data generator for the offboard computing device 700, which may instead communicate directly across the cloud network 900 with the remote computing device 800). Alternately, according to another embodiment the orthopedic device 100 may also be configured to communicate directly across the cloud network 900 with the remote computing device 800, without requiring the offboard computing device 700).

Beneficially here, any information generated by the orthopedic device 100 may then be stored in a cloud platform (backend server 870) which can then be reviewed and analyzed to assist in the recovery of the user 10. The information stored in the cloud server may also be provided via the web to a remote portal (third party device 850) where a third-party can also review the stored information, and/or it can be further processed to extract additional information and meaning. Further, the user (via offboard computing device 700 and/or personal device 810) and/or their healthcare provider (via third party device 850) may assess the information to make informed decisions.

Movement parameters, thresholds or limits selected can be determined by default numbers based on clinical knowledge from the medical industry, an overseeing medical provider such as a physician or physical therapist or custom parameters set by the user through an interface of offboard computing device 700. Because the information from the orthopedic device 100 can be remotely accessed, the medical professional can also set or adjust the scope of allowed movement. The software program optionally can have a motion capture feature wherein the user 10 performs a predetermined exercise and once the user 10 feels pain, the maximum level for a certain motion is then set. As the user 10 progresses to recovery, the exercise can be repeated to adjust the level appropriately.

The software program can also measure, monitor and track the movement of the body part over a period of time. This information can optionally be recorded and stored. The tracking can report the periods of use, the time the users' body part is in a correct or incorrect position. This can then be correlated to the user's activities and the user can learn the behaviors that may be causing the body part to not be in a correct position.

Parameters and angle measurements for movement are also useful in recovery. Post injury or operation, a medical professional will measure angles to determine the ability of the user the recovering body part. The angle of each joint will be measured as a starting point. Over time, new measurements are taken to make sure the user's function is improving with the end-goal of returning the user's function to their pre-operation or injured state. Optionally, goals can be set by an overseeing medical professional to encourage compliance.

By leveraging the massive data storage capacity of a cloud platform (backend server 870) and the high volume of precise, micromovement data captured by the orthopedic device 100, it will be possible to collect terabytes of data. This is a recovery of data that never has been accessible at this level of scale, diversity, and preciseness. The stored data, particularly if anonymized, will not only provide perspective of patients' continuous 7/24 motion data and recovery, but may also provide the protocols and recommendations from different providers. Further, being that the data is captured and managed with a single software application, it will be highly structured. With this amount of structured data, it may be possible to provide advances, data-centric correctional or remedial insights by applying deep learning techniques. In particular, the data may be parsed and dissected, drawing multi-dimensional correlations between patients, recovery methods, interventions, and outcomes. These learnings may then be applied to the data and to new patients, adapting their care automatically based on optimum recoveries seen by their relative cohort. Ultimately, a diagnostic AI may be created, which may advance recovery care, dynamically reacting to the patient progression and adapting the recovery on a daily basis while informing care givers and others.

In another embodiment the present disclosure is directed to a method for facilitating the care of a user. The method includes the steps of: a) providing the orthopedic device 100 b) creating a user account in a database; c) performing an initial examination of the user by an overseeing medical professional, whereby at least one medical record for the user is created; d) establishing a patient medical professional relationship; and e) monitoring the vitals and/or movements of the patient.

Optionally, and after providing the orthopedic device 100, the method may further include the steps of: a) accessing the database via the user account; b) requesting remote health care; and c) receiving remote health care, wherein the medical professional can (i) create a new medical record or update an existing medical record and the patient account in the database is automatically updated; (ii) instruct the user/patient to perform a task; (iii) adjust the functionality of the brace; and/or, (iv) review the patient's data recorded from the orthopedic device 100.

Beneficially, the combination of the user wearing the orthopedic device 100 and the tracking of data (e.g., via the system 1000) provided by the orthopedic device 100 provides more and continuous information of human movement and in real life activities outside artificial environment like a physical therapy office. The information is also a clinical tool for assessment and evaluation in human posture and movement, these systems can provide information about the vital signs of patients including but not limited to pulse, oxygenation, heart rate, hydration, temperature and the elderly. The information can also be utilized as a therapeutic or control system for orthotic or prosthetic devices to monitor various parameters, such as compliance, range of motion and magnitude of force. When a physician reviews the information they may alter the treatment program.

In yet another embodiment, the system 1000 software may include exercises. The exercises are again preset based on the user or patient's needs and most likely by a medical professional such as a physician or physical therapist. The user watches the exercises on the computing device's interface such as a mobile app running on the offboard computing device 700, and follows along conducting the exercises while wearing the orthopedic device 100. This optional motion capture feature can also be used to assist the user 10 in performing the exercises.

The software may then track the user's exercise and compliance. The information can then be reviewed by the healthcare professional. The healthcare professional upon review of the information may alter the treatment program such as changing the threshold or preset limits for movement giving the user more freedom. Or conversely if the user is not progressing or even getting worse, the preset threshold or limits may be further restricted. The same is true for the exercise—more improvement and the exercises provide for a broader range of motion.

According to one embodiment, the system 1000 may be used in telehealth applications. In particular, in a telehealth appointment, a healthcare professional may access the user's data over the cloud network 900 via the third party device 850. For example, the healthcare professional may follow along remotely with the patient (user 10) accessing stored data and prepared reports/analysis, and also view any tracking movement in real-time. Also for example, the healthcare professional may remotely direct the user to perform specific exercises and/or guide the user in a specific response/treatment, in view of the remotely accessed data of the user 10. Beneficially, the performance of exercises through the system in light of precise, real time data from the orthopedic device 100, may improve the healing or recovery process of the user 10 and/or be therapeutic, aiding the health of the user.

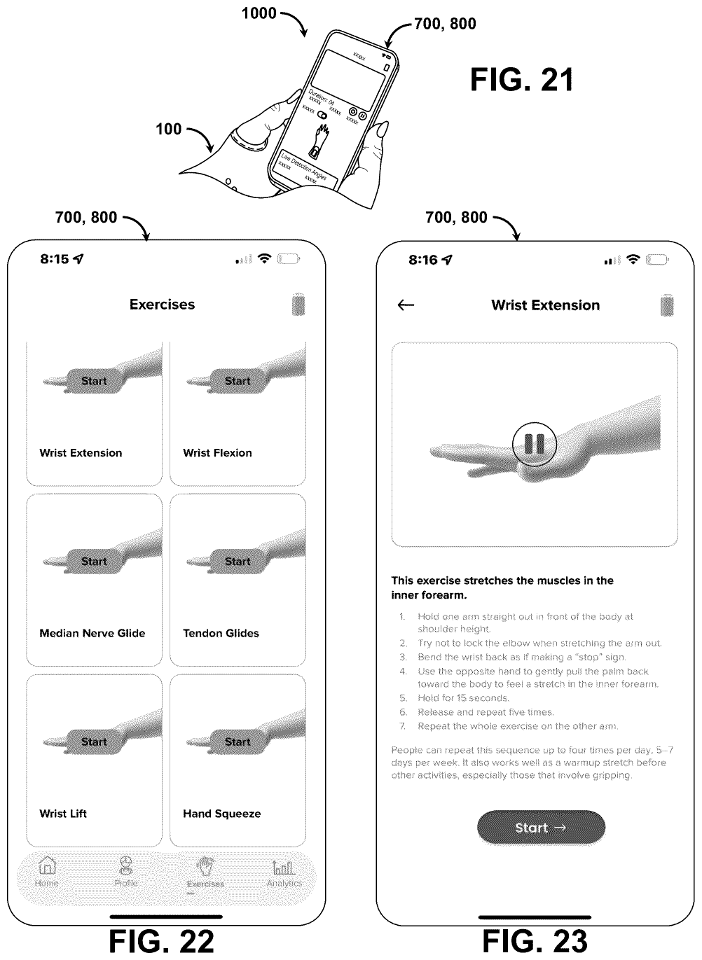

FIG. 21 an intelligent orthopedic device system in use, according to one embodiment of the present disclosure. As above, the system 1000 may include the orthopedic device 100 and an offboard computing device 700 and/or a remote computing device 800 configured to run a related software application. Preferably, and as shown, the system 1000 may include may include the orthopedic device 100 wirelessly connected to the offboard computing device 700 (here, a smart phone) of the user 10. As shown, the offboard computing device 700 is running a downloaded software application. In particular, the application is directed toward an exemplary patient rehabilitation protocol exercise, wherein the process is initiating.

In general, the software may be used to set the features and functionality of the orthopedic device 100, including the movement parameters or the correct and incorrect positions for the body part. For setup the user 10 will launch the software application, for example, on offboard computing device 700. In a preferred embodiment the software running on the computing device transmits via a wireless transceiver to the controller 500/processor 530 of the orthopedic device 100. The controller 500 confirms the setup with the software application. The software application registers the sensor data with the controller 500. The controller 500 confirms the registration with the software. The sensor 300 transmits the data to the software application and the software application processes the data. When the user 10 is finished, a disconnect signal can be transmitted to the software which then communicates to the controller 500 and the controller 500 confirms the disconnect.

The software is used to program or otherwise operate the processor 530. In one embodiment, the processor 530 is programmed to monitor the body parts movement and record the information. Movement is generally measured in degrees but can also be measured in radians. The parameters for a correct position and incorrect position can be initially set either by default numbers, or an overseeing medical professional. If a medical professional wanted to elect a different set of parameters the degrees of permitted and prohibited movement can be adjusted. Thus, when a user 10 exceeds the permissible movement it can trigger an alert. Alternatively, the software can display an open-source program wherein a pipe mirror image follows the user's movement. The user 10 can be asked to perform a series of exercises. Once the user 10 feels a certain pain level the movement stops and that becomes the highest parameter for movement. The goal being when the patient improves, this level will increase with time and the scope of permissible movements will expand.

The user 10 can also use a downloadable mobile application to interact with the software. The user's account and records are also stored in a physician or medical professional's portal (third party device 870) which can be accessed online. As an example, the medical professional would login to the software and then select the patient to view and the access all of the data and history collected by the device. This backend solution will allow for clinicians and patients to view data analytics as it pertains to progression, recovery compliance, and Device information. This enhances the value of the software application because it allows the coordination with any healthcare provider. And this would be helpful for the healthcare provider to provide to patients to compliment the patients therapy and recovery.

The user can also perform therapeutic exercises on their interface (e.g., display 552, offboard computing device 700, or personal device 810). This can then be reported remotely via the web to a portal (third party device 850). That portal can be then accessed by an overseeing medical professional so that person can observe the patient's exercise history, compliance and other information related.

The software can also be used to provide a complete physical therapy session. For example, an overseeing medical professional could assign a session time. During the session, the user's body part could be warmed for a period of time, then the exercise could be performed and tracked, with the session ending with a cooling period and TENS. The entire session is recorded and the medical professional and user could view the results of the session in the interface. Each session can be custom set depending on the user's needs and adjusted over time. As shown in the images, results of using the software can be plotted in graphs or charts to easily review the user's progress and compliance. Goals can also be set such as the user reaching a certain angle of movement and the user can view how far or close they are to the goal.

FIG. 22 is a smart phone screen shot of a user display for an intelligent orthopedic device system, according to one embodiment of the present disclosure. As shown, offboard computing device 700 is running a downloaded software application and has entered a selection of choices of patient rehabilitation protocol exercises via the user's mobile device.

FIG. 23 is a smart phone screen shot of a user display for an intelligent orthopedic device system, according to one embodiment of the present disclosure. Here, the “Wrist Extension” exercise has been selected. For each of the choices in the patient rehabilitation protocol exercises via a mobile device are purposes and instructions along with animation of the exercise prior to engaging in the specific exercise.

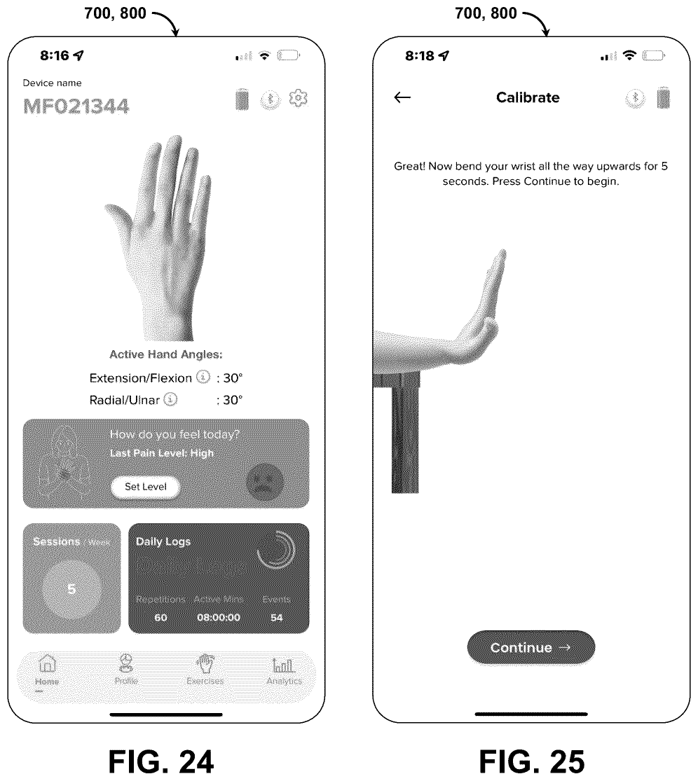

FIG. 24 is a smart phone screen shot of a user display for an intelligent orthopedic device system, according to one embodiment of the present disclosure. In particular, here is shown a home page summary, which provides live active mirroring of a patient's micro movements that their range of motions are measured. Also included is a pain survey questionnaire that allows the patient to provide their level of pain. As discussed above, the user 10 may use a much larger touch screen of the offboard computing device 700, being richer and more fully functioning than the limited display 552 of the orthopedic device 100. In this example, the home page provides summaries of patient rehabilitation protocols measuring their amount of sessions/week, daily repetitions, and active minutes. Finally, it measures the daily amount of alerts. All on a mobile device.

FIG. 25 is a smart phone screen shot of a user display for an intelligent orthopedic device system, according to one embodiment of the present disclosure. In particular, here is a calibration instructional screen with guided instructions and animation via mobile devices.

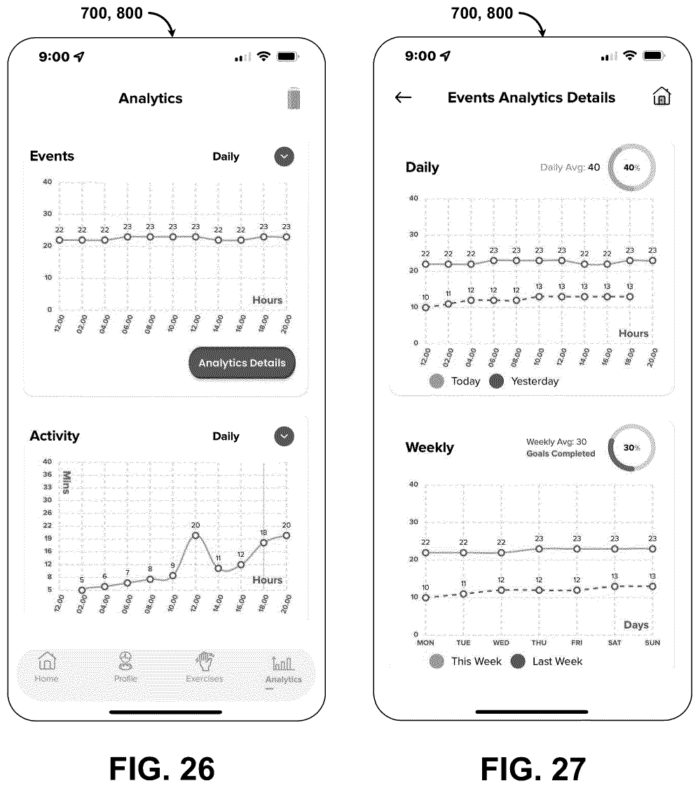

FIG. 26 is a smart phone screen shot of a user display for an intelligent orthopedic device system, according to one embodiment of the present disclosure. Here, the software application displays different graphical illustrations of the user's performance and progress over time. In particular, within the Analytics section on a mobile device a patient can review their measures of alerts, Sessions, Events, and Activity by daily, weekly, monthly, 6 months, and yearly.

FIG. 27 is a smart phone screen shot of a user display for an intelligent orthopedic device system, according to one embodiment of the present disclosure. Here, the software application displays different graphical illustrations of the user's performance and progress over time. In particular, the software application shows Events and Analytical Details on a mobile device, including a patients trends compared to time and against their goal.

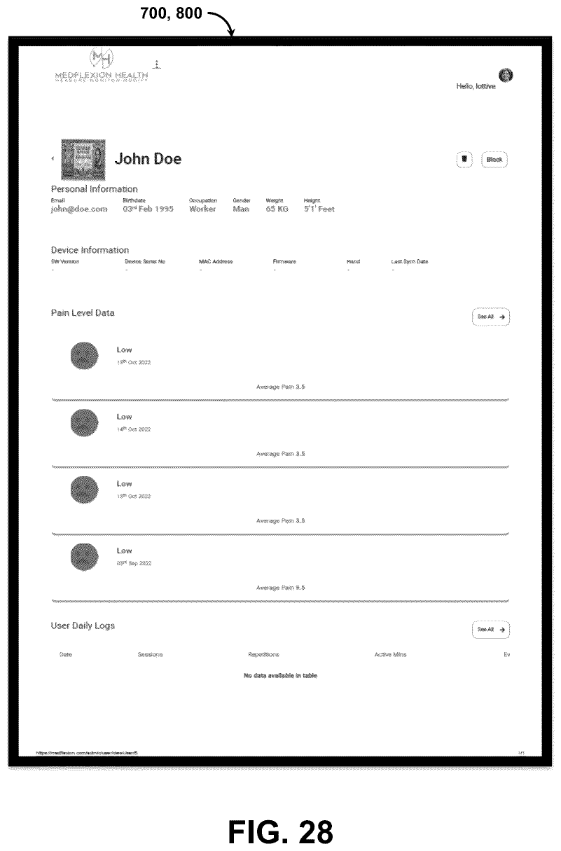

FIG. 28 is a computer screen shot of a user display for an intelligent orthopedic device system, according to one embodiment of the present disclosure. As shown, the personal device 810 (e.g., user's desktop computer) is running a downloaded software application and has entered through a cloud base backend and web portal that the patient or an approved caregiver can review the results of various parameters relative to clinical and rehabilitation results, personal information, device information and daily logs.

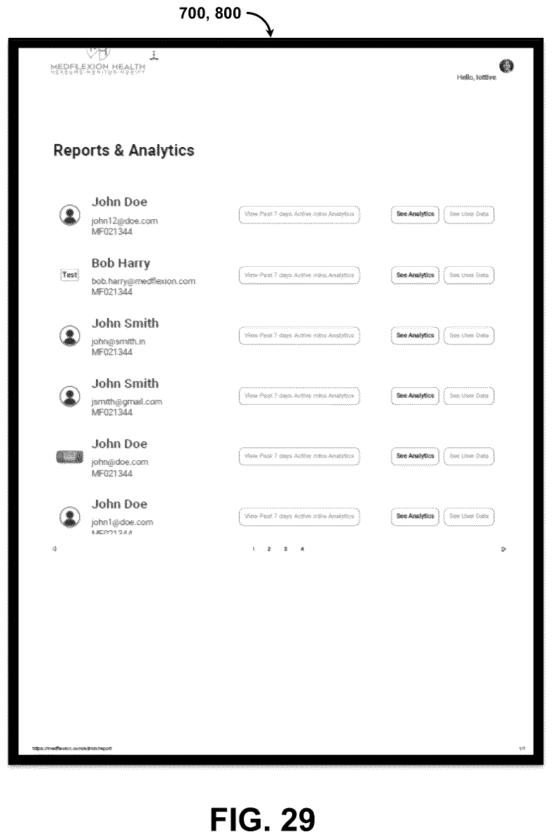

FIG. 29 is a computer screen shot of a user display for an intelligent orthopedic device system, according to one embodiment of the present disclosure. As shown, the third party device 810 (e.g., doctor's desktop computer) is running a downloaded software application and has entered through a cloud base backend and web portal a caregiver can review various patients' data.

For example, in the beginning of a recovery process the user's movement can be more restricted with additional freedom being allowed as the healing process progresses. It would also be useful to have reliable information regarding when the user or patient has improved so that the treatment protocol can be adjusted accordingly. A brace that provides therapeutic properties can also be also desirable. Telehealth or telemedicine has become increasingly popular, and sometimes 3 necessary, so a brace that can provide information to an overseeing medical professional remotely can provide additional benefits. Thus, it is desirable to have an intelligent brace system where a real-time assessment of the patient's current state can be determined, the recovery can be monitored, the activity measured, behaviors modified all the while providing other therapeutic benefits and in constant communication with the user and a healthcare provider.

For these reasons there is a need for alternative brace coupled with a system that improves the patient and medical provider's knowledge of the musculoskeletal issue, that can be adjusted to address the present phase of the treatment and be used to improve the preventive or recovery process.

The user can then learn from the program what behaviors cause the body part to not be in the selected position. In use, the user can optionally use therapeutic features to aid in recovery. The user can use software program to participate in predetermined exercises for the body part and monitor the body part's movements during those exercises. Motion capture can be optionally used by the user in the performance of the exercises.

In another embodiment the device comprises a warming element. The processor can be programmed to produce an increase in temperature for the body mount 200 such that the body part is warmed.

In still another embodiment the device comprises tens. The processor can be programmed to produce an electric therapeutic shock to a desired location on the body.

In yet another embodiment the present disclosure comprises a method for using the intelligent brace system. The method comprises a user wearing a brace comprising a processor in communication with a software program that has preset information to assist in the measuring and monitoring the position of a users' body part using one or more than one sensors on a body mount 200 worn by the user. In use, the processor in connection with the sensor/s determine whether the body part is in a correct or incorrect position. monitors and tracks the user's movements and vitals in real time. Algorithms programmed in a software application can record whether a user's motion is an optimal, borderline or poor position. This information can be collected and stored over a period of time such that the user and/or their medical professional can review the information. Optionally, the device is programmed to set parameters of a desired or selected position for the body part for a specific patient.

Embodiments of the subject matter and the functional operations described in this specification can be implemented in digital electronic circuitry, or in computer software, firmware, hardware, resident software, microcode, etc., including the structures disclosed in this specification and their structural equivalents, or in combinations of one or more of them. Further, the processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application-specific integrated circuit).

Aspects of the system and method can take the form of a computer program product accessible from a computer-usable or computer-readable medium providing program code for use by or in connection with a computer or any instruction execution system. For the purposes of this description, a computer-usable or computer readable medium can be any apparatus that can contain, store, communicate, propagate, or transport the program for use by or in connection with the instruction execution system, apparatus, or device. Further, an application program may be uploaded to, and executed by, a machine comprising any suitable architecture. Preferably, the machine is implemented on a computer platform having hardware such as one or more central processing units (“CPUs”), a memory, and input or output interfaces.

The medium can be an electronic, magnetic, optical, electromagnetic, infrared, or semiconductor system (or apparatus or device) or a propagation medium. Examples of a computer readable medium comprise a semiconductor or solid-state memory, magnetic tape, a removable computer diskette, a random access memory (RAM), a read-only memory (ROM), a rigid magnetic disk and an optical disk. Current examples of optical disks comprise compact disk-read only memory (CD-ROM), compact disk-read/write (CD-R/W) and DVD.

A data processing system suitable for storing and/or executing program code comprises at least one processor coupled directly or indirectly to memory elements through a system bus. The memory elements can include local memory employed during actual execution of the program code, bulk storage, and cache memories that provide temporary storage of at least some program code in order to reduce the number of times code is retrieved from bulk storage during execution. Input/output or I/O devices (including but not limited to keyboards, displays, pointing devices, etc.) can be coupled to the system either directly or through intervening I/O controllers. Network adapters may also be coupled to the system to enable the data processing system to become coupled to other data processing systems or remote printers or storage devices through intervening private or public networks. Modems, cable modem and Ethernet cards are just a few of the currently available types of network adapters.

Described above, aspects of the present application are embodied in a World Wide Web (“WWW”) or (“Web”) site accessible via the Internet. As is well known to those skilled in the art, the term “Internet” refers to the collection of networks and routers that use the Transmission Control Protocol/Internet Protocol (“TCP/IP”) to communicate with one another. The internet can include a plurality of local area networks (“LANs”) and a wide area network (“WAN”) that are interconnected by routers. The routers are special purpose computers used to interface one LAN or WAN to another. Communication links within the LANs may be wireless, twisted wire pair, coaxial cable, or optical fiber, while communication links between networks may utilize 56 Kbps analog telephone lines, 1 Mbps digital T-1 lines, 45 Mbps T-3 lines or other communications links known to those skilled in the art. Furthermore, computers and other related electronic devices can be remotely connected to either the LANs or the WAN via a digital communications device, modem and temporary telephone, or a wireless link. It will be appreciated that the internet comprises a vast number of such interconnected networks, computers, and routers. The Internet has recently seen explosive growth by virtue of its ability to link computers located throughout the world. As the Internet has grown, so has the WWW. As is appreciated by those skilled in the art, the WWW is a vast collection of interconnected or “hypertext” documents written in HTML, or other markup languages, that are electronically stored at or dynamically generated by “WWW sites” or “Web sites” throughout the Internet. Additionally, client-side software programs that communicate over the Web using the TCP/IP protocol are part of the WWW, such as JAVA® applets, instant messaging, email, browser plug-ins, Macromedia Flash, chat and others. Other interactive hypertext environments may include proprietary environments such as those provided in America Online or other online service providers, as well as the “wireless Web” provided by various wireless networking providers, especially those in the cellular phone industry. It will be appreciated that the present application could apply in any such interactive communication environments, however, for purposes of discussion, the Web is used as an exemplary interactive hypertext environment with regard to the present application.

A website is a server/computer connected to the Internet that has massive storage capabilities for storing hypertext documents and that runs administrative software for handling requests for those stored hypertext documents as well as dynamically generating hypertext documents. Embedded within a hypertext document are a number of hyperlinks, i.e., highlighted portions of text which link the document to another hypertext document possibly stored at a website elsewhere on the Internet. Each hyperlink is assigned a URL that provides the name of the linked document on a server connected to the Internet. Thus, whenever a hypertext document is retrieved from any web server, the document is considered retrieved from the World Wide Web. Known to those skilled in the art, a web server may also include facilities for storing and transmitting application programs, such as application programs written in the JAVA® programming language from Sun Microsystems, for execution on a remote computer. Likewise, a web server may also include facilities for executing scripts and other application programs on the web server itself.

A remote access user may retrieve hypertext documents from the World Wide Web via a web browser program. A web browser, such as Netscape's NAVIGATOR® or Microsoft's Internet Explorer, is a software application program for providing a user interface to the WWW. Upon request from the remote access user via the web browser, the web browser requests the desired hypertext document from the appropriate web server using the URL for the document and the hypertext transport protocol (“HTTP”). HTTP is a higher-level protocol than TCP/IP and is designed specifically for the requirements of the WWW. HTTP runs on top of TCP/IP to transfer hypertext documents and user-supplied form data between server and client computers. The WWW browser may also retrieve programs from the web server, such as JAVA applets, for execution on the client computer. Finally, the WWW browser may include optional software components, called plug-ins, that run specialized functionality within the browser.

One or more embodiments of the present disclosure may be implemented with one or more computer readable media, wherein each medium may be configured to include thereon data or computer executable instructions for manipulating data. The computer executable instructions include data structures, objects, programs, routines, or other program modules that may be accessed by a processing system, such as one associated with a general purpose computer or processor capable of performing various different functions or one associated with a special purpose computer capable of performing a limited number of functions. Computer executable instructions cause the processing system to perform a particular function or group of functions and are examples of program code means for implementing steps for methods disclosed herein. Furthermore, a particular sequence of the executable instructions provides an example of corresponding acts that may be used to implement such steps. Examples of computer readable media include random-access memory (“RAM”), read-only memory (“ROM”), programmable read-only memory (“PROM”), erasable programmable read-only memory (“EPROM”), electrically erasable programmable read-only memory (“EEPROM”), compact disk read-only memory (“CD-ROM”), or any other device or component that is capable of providing data or executable instructions that may be accessed by a processing system. Examples of mass storage devices incorporating computer readable media include hard disk drives, magnetic disk drives, tape drives, optical disk drives, and solid state memory chips, for example. The term processor as used herein refers to a number of processing devices including personal computing devices, mobile phones, servers, general purpose computers, special purpose computers, application-specific integrated circuit (ASIC), and digital/analog electronic circuits with discrete components, for example.

The above description of the various embodiments has been presented for the purposes of illustration and description and is provided to enable a person of ordinary skill in the art to make or use the subject matter of the disclosure. Various modifications to the embodiments will be readily apparent to those skilled in the art, and the generic principles defined herein may be applied to other variations without departing from the spirit or the scope of this disclosure, and it is not intended to be exhaustive nor to limit the disclosure to the precise form disclosed. Thus, it is to be understood that the disclosure is not intended to be limited to the examples and designs described herein, which merely represent a presently preferred implementation of the disclosure, but that the disclosure is to be accorded the widest scope consistent with the principles and novel features disclosed herein. It is to be further understood that the scope of the present disclosure fully encompasses other embodiments that may become obvious to those skilled in the art in view of this detailed description, the drawings, and the claims, as well as any equivalents to the same.

1. An intelligent orthopedic device for a jointed body part of a user, the jointed body part including a first member and a second member joined together by a joint, the intelligent orthopedic device comprising:
a body mount configured to attach to the user proximate the jointed body part of the user;
a body sensor coupled to the body mount, said body sensor configured to provide a metric of an angular displacement between the first member and the second member of the jointed body part;
a position resistor coupled to the body mount, said position resistor configured to apply a force opposite said angular displacement to the jointed body part; and
a controller coupled to the body mount and communicably coupled to the body sensor, the controller configured to receive the metric of the angular displacement from the body sensor and to operate the position resistor to apply said force opposite the angular displacement to the jointed body part in response to the metric of the angular displacement exceeding a predefined threshold.

2. The intelligent orthopedic device of claim 1 further comprising an additional body sensor coupled to the body mount and communicably coupled to the controller, said additional body sensor configured to provide at least one of biometrics, a pulse, a blood oxygenation, a heart rate, a hydration, and a temperature of the user to the controller.

3. The intelligent orthopedic device of claim 1, wherein the position resistor includes at least one cavity configured to house an electrorheological fluid, said at least one cavity arranged about the body mount such that activation of the electrorheological fluid stiffens the intelligent orthopedic device about the jointed body part against the angular displacement; and
wherein the controller is further configured to activate and deactivate the electrorheological fluid of the position resistor.

4. The intelligent orthopedic device of claim 1, wherein the position resistor includes at least electromechanical stiffener configured to stiffen the intelligent orthopedic device about the jointed body part against the angular displacement; and
wherein the controller is further configured to activate and deactivate the at least electromechanical stiffener of the position resistor.

5. The intelligent orthopedic device of claim 1, further comprising at least one stiffener insert; and
wherein the body mount includes a stiffener interface, said stiffener interface configured to removably receive the stiffener insert, and to transfer loading between the jointed body part and the stiffener insert so as to limit the angular displacement of the jointed body part when the stiffener insert is received in the stiffener interface of the body mount.

6. The intelligent orthopedic device of claim 1, further comprising a thermal interface arranged to be in thermal communication with a predefined body part of the user, said thermal interface configured modify a temperature of said predefined body part, said thermal interface including at least one heating element coupled to the body mount, said at least one heating element configured to warm the predefined body part; and
wherein the controller is further configured to operate the thermal interface, and to power the at least one heating element of the thermal interface.

7. The intelligent orthopedic device of claim 1, further comprising a thermal interface arranged to be in thermal communication with a predefined body part of the user, said thermal interface configured modify a temperature of said predefined body part, said thermal interface including at least one cooling element coupled to the body mount, said at least one cooling element configured to cool the predefined body part; and
wherein the controller is further configured to operate the thermal interface, and to power the at least one cooling element of the thermal interface.

8. The intelligent orthopedic device of claim 1, further comprising one or more Transcutaneous Electrical Neuromuscular Stimulation (“TENS”) elements anchored to and positioned about the body mount at one or more predefined locations of the jointed body part of a user; and
wherein the controller is configured to operate the one or more TENS elements to apply electrical stimulation to said one or more predefined locations of the jointed body part.

9. The intelligent orthopedic device of claim 1, wherein the jointed body part of the user includes a wrist of the user, and the body mount is adapted as a flexible wrist brace.

10. The intelligent orthopedic device of claim 1, further comprising a user interface having a user output, said user output configured to issue alert signals to the user at least one of visually, aurally, and haptically, in response to the jointed body part of the user exceeding a preset threshold limit.

11. The intelligent orthopedic device of claim 1, further comprising a user interface having a user input, said user input configured to receive feedback signals from the user; and
wherein the controller includes a power supply, a processor powered by the power supply, and a memory communicably coupled to the processor, the processor configured to record in the memory the feedback signals received by user input together with at least the metric of the angular displacement from the body sensor.

12. The intelligent orthopedic device of claim 1, wherein the controller includes a power supply, a processor powered by the power supply, a memory communicably coupled to the processor, and a communication port communicably coupled to the processor, the controller further configured to record metrics of the angular displacement from the body sensor over time, in the memory, and to transmit said metrics of the angular displacement remotely from the controller via the communication port.

13. The intelligent orthopedic device of claim 12, further comprising:
an additional body sensor coupled to the body mount and communicably coupled to the controller, said additional body sensor configured to provide at least one of biometrics, a pulse, a blood oxygenation, a heart rate, a hydration, and a temperature of the user to the controller;
at least one stiffener insert;
a thermal interface arranged to be in thermal communication with a predefined body part of the user, said thermal interface configured modify a temperature of said predefined body part, said thermal interface including at least one heating element coupled to the body mount, said at least one heating element configured to warm the predefined body part, said thermal interface further including at least one cooling element coupled to the body mount, said at least one cooling element configured to cool the predefined body part;
one or more Transcutaneous Electrical Neuromuscular Stimulation (“TENS”) elements anchored to and positioned about the body mount at one or more predefined locations of the jointed body part of a user; and
a user interface having a user output and a user input, said user output configured to issue alert signals to the user at least one of visually, aurally, and haptically, in response to the jointed body part of the user exceeding a preset threshold limit, said user input configured to receive feedback signals from the user; and
wherein the position resistor includes at least one cavity configured to house an electrorheological fluid, said at least one cavity arranged about the body mount such that activation of the electrorheological fluid stiffens the intelligent orthopedic device about the jointed body part against the angular displacement;
wherein the controller is further configured to activate and deactivate the electrorheological fluid of the position resistor;
wherein the body mount includes a stiffener interface, said stiffener interface configured to removably receive the stiffener insert, and to transfer loading between the jointed body part and the stiffener insert so as to limit the angular displacement of the jointed body part when the stiffener insert is received in the stiffener interface of the body mount;
wherein the controller is further configured to operate the thermal interface, and to power the at least one heating element of the thermal interface;
wherein the controller is further configured to operate the thermal interface, and to power the at least one cooling element of the thermal interface;
wherein the controller is further configured to operate the one or more TENS elements to apply electrical stimulation to said one or more predefined locations of the jointed body part;
wherein the jointed body part of the user includes a wrist of the user, and the body mount is adapted as a flexible wrist brace;
wherein the controller is further configured to record metrics of the angular displacement from the body sensor over time, in the memory, and to transmit said metrics of the angular displacement remotely from the controller via the communication port; and
wherein the processor is configured to record in the memory the feedback signals received by user input together with at least the metric of the angular displacement from the body sensor.

14. An intelligent orthopedic device system for a jointed body part of a user, the jointed body part including a first member and a second member joined together by a joint, the intelligent orthopedic device system comprising:
an intelligent orthopedic device including
a body mount configured to attach to the user proximate the jointed body part of the user,
a body sensor coupled to the body mount, said body sensor configured to provide a metric of an angular displacement between the first member and the second member of the jointed body part,
a position resistor coupled to the body mount, said position resistor configured to apply a force opposite said angular displacement to the jointed body part, and
a controller coupled to the body mount and communicably coupled to the body sensor, the controller configured to receive the metric of the angular displacement from the body sensor and to operate the position resistor to apply said force opposite the angular displacement to the jointed body part in response to the metric of the angular displacement exceeding a predefined threshold; and
an offboard computing device communicably coupled to the controller of the intelligent orthopedic device, and configured to host and run a related software application, said software application programmed to receive and send information regarding one or more features and functions of the intelligent orthopedic device including at least one of measuring, monitoring, tracking and storing the angular displacement between the first member and the second member of the jointed body part.

15. The intelligent orthopedic device system of claim 14, wherein the offboard computing device is a handheld mobile device operable by the user in real time, and is communicably coupled to the controller of the intelligent orthopedic device via a wireless communication radio link.

16. The intelligent orthopedic device system of claim 15, wherein the offboard computing device is further communicably coupled to a remote computing device via an Internet connection the remote computing device configured to receive the information regarding one or more features and functions of the intelligent orthopedic device.

17. The intelligent orthopedic device system of claim 15, further comprising a backend server communicably coupled to offboard computing device via an Internet connection, the backend server configured to receive and store the information regarding one or more features and functions of the intelligent orthopedic device.

18. The intelligent orthopedic device system of claim 14, wherein the intelligent orthopedic device further includes:
an additional body sensor coupled to the body mount and communicably coupled to the controller, said additional body sensor configured to provide at least one of biometrics, a pulse, a blood oxygenation, a heart rate, a hydration, and a temperature of the user to the controller;
at least one stiffener insert;
a thermal interface arranged to be in thermal communication with a predefined body part of the user, said thermal interface configured modify a temperature of said predefined body part, said thermal interface including at least one heating element coupled to the body mount, said at least one heating element configured to warm the predefined body part, said thermal interface further including at least one cooling element coupled to the body mount, said at least one cooling element configured to cool the predefined body part;
one or more Transcutaneous Electrical Neuromuscular Stimulation (“TENS”) elements anchored to and positioned about the body mount at one or more predefined locations of the jointed body part of a user; and
a user interface having a user output and a user input, said user output configured to issue alert signals to the user at least one of visually, aurally, and haptically, in response to the jointed body part of the user exceeding a preset threshold limit, said user input configured to receive feedback signals from the user; and
wherein the controller includes a power supply, a processor powered by the power supply, a memory communicably coupled to the processor, and a communication port communicably coupled to the processor, the controller further configured to record metrics of the angular displacement from the body sensor over time, in the memory, and to transmit said metrics of the angular displacement remotely from the controller via the communication port;
wherein the position resistor includes at least one cavity configured to house an electrorheological fluid, said at least one cavity arranged about the body mount such that activation of the electrorheological fluid stiffens the intelligent orthopedic device about the jointed body part against the angular displacement;
wherein the controller is further configured to activate and deactivate the electrorheological fluid of the position resistor;
wherein the body mount includes a stiffener interface, said stiffener interface configured to removably receive the stiffener insert, and to transfer loading between the jointed body part and the stiffener insert so as to limit the angular displacement of the jointed body part when the stiffener insert is received in the stiffener interface of the body mount;
wherein the controller is further configured to operate the thermal interface, and to power the at least one heating element of the thermal interface;
wherein the controller is further configured to operate the thermal interface, and to power the at least one cooling element of the thermal interface;
wherein the controller is further configured to operate the one or more TENS elements to apply electrical stimulation to said one or more predefined locations of the jointed body part;
wherein the jointed body part of the user includes a wrist of the user, and the body mount is adapted as a flexible wrist brace;
wherein the controller is further configured to record metrics of the angular displacement from the body sensor over time, in the memory, and to transmit said metrics of the angular displacement remotely from the controller via the communication port; and
wherein the processor is configured to record in the memory the feedback signals received by user input together with at least the metric of the angular displacement from the body sensor.

19. An intelligent orthopedic device for a jointed body part of a user, the jointed body part including a first member and a second member joined together by a joint, the intelligent orthopedic device comprising:
a body mount configured to attach to the user proximate the jointed body part of the user;
a body sensor coupled to the body mount, said body sensor configured to provide a metric of an angular displacement between the first member and the second member of the jointed body part;
a means to apply at least one of cooling, warming, and Transcutaneous Electrical Neuromuscular Stimulation (“TENS”) to the jointed body part of the user, said means coupled to the body mount; and
a controller coupled to the body mount and communicably coupled to the body sensor, the controller configured to receive the metric of the angular displacement from the body sensor, to operate said means, and to apply said at least one of cooling, warming, and “TENS” to the jointed body part of the user via said means, in response to the metric of the angular displacement exceeding a predefined threshold.

20. The intelligent orthopedic device of claim 1, further comprising a user interface having a user input, said user input configured to receive feedback signals from the user; and
wherein the controller includes a power supply, a processor powered by the power supply, and a memory communicably coupled to the processor, the processor configured to associate in the memory the feedback signals received by user input together with the operation of said means.
